(19) United States
(12) Patent Application Publication
(10) Pub. No.: US 2008/0312198 A1
(43) Pub. Date: Dec. 18, 2008

(54) PHARMACEUTICAL PRODUCTS CONTAINING HORMONES AND A 25-HYDROXY VITAMIN D COMPOUND

(76) Inventor: Gustavo C. RODRIGUEZ, Durham, NC (US)

Correspondence Address: JENNER & BLOCK, LLP, ONE IBM PLAZA, CHICAGO, IL, 60611, US

(21) Appl. No.: 11/782,067

(22) Filed: Jul. 24, 2007

Related U.S. Application Data
(63) Continuation-in-part of application No. 11/763,250, filed on Jun. 14, 2007.

(51) Int. Cl. A61K 31/59; A61P 3/14

(52) U.S. Cl. 514/167

(57) ABSTRACT

The present invention relates to pharmaceutical products containing progestins in combination with the 25 hydroxy Vitamin D compounds. The 25 hydroxy Vitamin D compounds are preferably administered with the progestins. In OC and HRT regimens, the 25 hydroxy Vitamin D compounds can be administered daily, or on a non-daily basis, and if so, preferably when the progestin dosages are the highest in the cycle.

This application is a continuation-in-part of Ser. No. 11/763,250 filed Jun. 14, 2007.

### FIELD OF THE INVENTION

The present invention relates generally to pharmaceutical products containing progestins in combination with 25-hydroxy Vitamin D compounds.

### BACKGROUND OF THE INVENTION

Vitamin D is a fat soluble vitamin which is essential as a positive regulator of calcium homeostasis. In the skin 7-Dehydrocholesterol (pro-Vitamin D3) is photolyzed by ultraviolet light to pre-Vitamin D3, which spontaneously isomerizes to Vitamin D3 (cholecalciferol). Vitamin D3 production via endogenous production by skin requires exposure of the skin to direct UV sunlight. Endogenous production is thus decreased in northern latitudes, where UV sunlight is more tangential, and also in individuals with pigmented skin, which inhibits UV absorption. The skin has the capacity to produce large amounts of Vitamin D3. With prolonged exposure of most skin surfaces to direct UV light, the skin is capable of producing thousands of International Units of Vitamin D3 per day. Maximum daily production plateaus however at approximately 10,000 IU/day, due to protective mechanisms designed to prevent D3 excess. As Vitamin D3 is produced in the skin, a portion of it undergoes metabolic degradation in response to UV light. At high levels of daily synthesis (beyond 10,000 IU), the rate of synthesis and breakdown of Vitamin D3 in the skin reaches an equilibrium.

Vitamin D3 (cholecalciferol), the structure of which is set out below, is converted into an active hormone by hydroxylation reactions occurring in the liver to produce 25-hydroxyvitamin D3 (“25(OH)D3” or “calcidiol”), which is then converted in the kidneys via the 1-alpha hydroxylase enzyme to produce 1,25-dihydroxyvitamin D3 (“1,25-dihydroxycholecalciferol” or “calcitriol” or “1,25(OH)2D3”). The beneficial effects of Vitamin D are due to the activity of 1,25 (OH)2 D3, the active form of the molecule. Vitamin D3, 25-hydroxyvitamin D3 and 1,25-dihydroxyvitamin D3 are shown below:

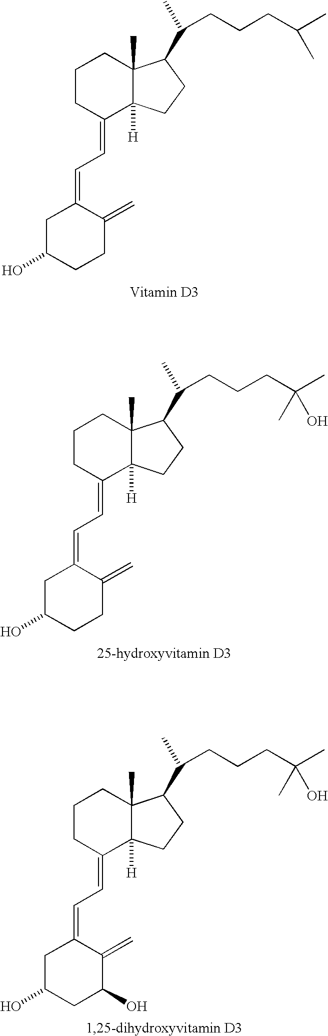

The active form of Vitamin D (1,25 (OH)2 D3) in circulation is primarily derived by 1-alpha hydroxylation of circulating 25(OH)D3 in the kidney. Circulating 25(OH)D3 is the substrate for the 1-alpha hydroxylase enzyme in kidney tubules, which converts 25(OH)D3 to 1,25 (OH)2 D3. As compared to other tissues, the kidney has the capacity for enhanced absorption of 25(OH)D3 and thus production of 1,25 (OH)2 D3. After subsequent hydroxylation by the renal 1-alpha hydroxylase enzyme, 1,25 (OH)2 D3 is produced by the kidney in amounts sufficient to have systemic effects. The renal 1-alpha hydroxylase is highly regulated (by PTH, calcium, and 1,25 (OH)2 D3) such that production of 1,25 (OH)2 D3 by the kidney is tightly controlled to limit excess and thus 1,25 (OH)2 D3 circulating levels are generally maintained within a tight range. 1,25 (OH)2 D3 promotes calcium absorption in the gut, and inhibits PTH.

The 1-alpha hydroxylase enzyme is also expressed in non-renal sites, including the breast, prostate, ovary, colon, and cells of the immune system. Circulating 25 (OH)D3 is a substrate for conversion to 1,25 (OH)2 D3 via the 1-alpha hydroxylase enzyme at these sites. As compared to the kidney, non renal tissues have limited capacity to absorb 25 (OH)D3. With the exception of certain disease states, non renal tissues cannot produce enough of the active form of the Vitamin to have a systemic effect. The extrarenal 1-alpha hydroxylase enzyme is not under the tight regulation typical of its renal counterpart. The extrarenal 1-alpha hydroxylase enzyme is not negatively regulated by changes in 1,25 (OH)2 D3, PTH, or calcium as is the renal enzyme.

Calcitriol (1,25 (OH)2 D3) is the form of vitamin D3 that is responsible for the majority of the bone-related benefits and non-skeletal health benefits of the vitamin for prevention of cancer and other chronic diseases. 1,25 (OH)2 D3 (calcitriol) is available for direct oral administration to patients. The half life after oral administration is short (5-8 hours). Despite the short half life of 1,25 (OH)2 D3, there are safety concerns with routine administration. At doses of 1,25 (OH)2 D3 of 0.5 micrograms per day or even 5 micrograms per week that have been demonstrated to enhance bone health, 1-3% of individuals develop hypercalcemia. Moreover, because the kidney tightly regulates production of 1,25 (OH)2 D3, the renal production of 1,25 (OH)2 D3 will be suppressed when circulating levels of 1,25 (OH)2 D3 are high. Thus, while oral administration of 1,25 (OH)2 D3 will increase peak circulating levels of 1,25 (OH)2 D3, the result will be less production in the kidney and less of an impact on steady state 1,25 (OH)2 D3 levels. Moreover, this increase in 1,25 D levels will occur at the risk of hypercalcemia. Serum levels of 1,25-dihydroxyvitamin D3 are closely regulated and typically range from 15-60 pg/mL. Serum 1,25-dihydroxyvitamin D3 has a half-life of 6-8 hours. 1,25-dihydroxyvitamin D3 partitions into cells by virtue of its lipophilicity, binds to intracellular receptors, and translocates to the nucleus where the vitamin-receptor complex controls the transcription of a number of genes, many of which relate to calcium metabolism. Corder et al., Cancer Epidemiology, Biomarkers & Prevention 2:467-472 (1993).

Serum levels of 25-hydroxyvitamin D3 are not closely regulated and will rise with increased sun exposure or oral intake of D3. Blood levels typically range from 15 to 80 ng/mL. In addition to 25-hydroxyvitamin D3 produced in the liver, 25-hydroxyvitamin D3 (“calcidiol”) has also been available for direct oral administration to patients. The half life of 25 (OH) D3 after oral administration is approximately 2 weeks.

Of interest to the present invention is the disclosure of applicant's U.S. Pat. No. 6,028,064 entitled “Prevention of Ovarian Cancer by Administration of Progestin Products.” That patent discloses a method for preventing the development of epithelial ovarian cancer by administering progestin products, either alone or in combination with other agents, such as estrogen products. Specifically, a method is described for preventing ovarian cancer comprising administering to a female subject an amount of progestin product effective to increase apoptosis in ovarian epithelial cells of the female subject. Also of interest are Rodriguez et al. U.S. Pat. Nos. 6,034,074, 6,407,082 and 6,444,658 and 7,053,074. The patents include disclosures of methods and compositions for increasing apoptosis in non-neoplastic ovarian epithelial cells of female subjects by administering Vitamin D compounds in amounts effective to induce apoptosis of non-neoplastic epithelial cells. The patents further include disclosures of oral contraceptive and hormone replacement therapy products with Vitamin D compounds.

U.S. Pat. Nos. 6,034,074, 6,407,082, 6,444,658 and 7,053,074 describe the term “Vitamin D compound”, including “Vitamin D”, “Vitamin D analogue”, or “Vitamin D derivative” as used therein as including any compound which activates the Vitamin D receptor, by binding or otherwise, either in its form of administration or in a form to which it is converted by processing by the human body. Those patents state that suitable analogues and derivatives are expected to include but are not limited to the following: 1α-hydroxyvitamin D3; 25-hydroxyvitamin D3; 1,24,25-(OH)3D3; 24,25-(OH)2D3; 1,25,26-(OH)3D3; 24,25-(OH)2D3; 1,25-dihydroxy-16-ene-23-yne-26, 27-hexafluorocholecalciferol; 25,26-dehydro-1a,24R-dihydroxycholecalciferol and 25,26-dehydro-1a,24S-dihydroxycholecalciferol; 1a-hydroxy-19-nor-vitamin D analogues; 26,28-methylene-1a,25-dihydroxyvitamin D2 compounds; 1a-hydroxy-22-iodinated vitamin D3 compounds; 23-Oxa-derivatives of Vitamin D; and fluorinated Vitamin D analogues; 20-methyl-substituted Vitamin D derivatives; (E)-20(22)-Dehydrovitamin D compounds; 19-nor-Vitamin D3 compounds with substituents at the 2-position; and 22-thio Vitamin D derivatives. Those patents teach that preferred dosages of the Vitamin D compound effective to increase apoptosis of non-neoplastic ovarian epithelial cells range from 0.0001 to 1.0 mcg/kg of body weight (based upon the apoptotic potency of 1,25-dihydroxyvitamin D3) with dosages ranging from about 0.005 to 0.75 mcg/kg being more preferred and dosages of about 0.05 to 0.5 mcg/kg being particularly preferred. (A typographical error of “mg” rather than “mcg” appears in three of the patents). Those patents also state that prophylactic regimens for administration of Vitamin D compounds for normal female individuals and for those at increased risk of ovarian epithelial cancer can include daily or other periodic administration of Vitamin D compounds. The patents also state that it is contemplated that preferred regimens for prevention of ovarian cancer may comprise periodic administration of relatively larger dosages of Vitamin D compounds on a monthly or less than monthly basis rather than more frequent administration. Those patents also teach that the larger dosage would preferably range from a dosage equivalent to at least 400 IU, more preferably a dosage equivalent to at least 2000 IU, or still more preferably a dosage equivalent to 4000 IU (40 IU equals 1 mcg).

Organon previously sold 25-hydroxy Vitamin D3 by prescription under the trade name Calderol® (Organon, West Orange, N.J.). However, the product was discontinued. One commentator noted that Organon's discontinuation of 25(OH)D may have made sense due to the availability of Vitamin D₃ itself and its ability to increase plasma 25(OH)D concentrations. Veith, The Pharmacology Of Vitamin D, Including Fortification Strategies, Chapter 61, In: Feldman D, Pike J W, Glorieux F H, editors. Vitamin D. 2nd ed. New York: Elsevier Academic Press; 2005. Applicant's invention is based on the belief that administration of 25-hydroxy Vitamin D3 provides enhanced benefits as compared to both Vitamin D3 and 1,25 di-hydroxy Vitamin D3 when combined with progestins.

### SUMMARY OF THE INVENTION

The present invention provides pharmaceutical products containing the 25 hydroxy metabolite of Vitamin D compounds at specified dosages and according specified schedules. The present invention further includes compositions, regimens and methods combining 25-hydroxy Vitamin D compounds with progestins.

The present invention provides hormonal products containing 25-hydroxy Vitamin D3 at dosages and schedules designed to ensure maximum safety, while at the same time achieving optimal serum levels of 25 (OH) D3 to confer both skeletal and non skeletal benefits of Vitamin D. These optimal dosages and schedules include the highest dosages of vitamin D that are possible without causing significant side effects. Furthermore, it is anticipated by the inventor that combinations of 25-hydroxy Vitamin D compounds with hormonal products such as progestins will confer unique benefits as compared to those hormonal products administered alone, with these benefits related to an enhanced effect of vitamin D over vitamin D administered alone.

While not wished to be bound by any particular theory, the inventor believes that the combination of vitamin D with progestins causes synergistic inhibition of cell viability in cells derived from the human ovarian epithelium. Furthermore, the inventor has discovered that progestins cause inhibition and/or degradation of 24 hydroxylase, the enzyme that causes 1,25-dihydroxy Vitamin D, and other 25 hydroxylated Vitamin D compounds, to become inactive. It is thus contemplated that adding a progestin to vitamin D will prolong the local half life of 25-hydroxy Vitamin D compounds in the ovarian epithelium and other sites in the body (via inhibition/degradation of 24 hydroxylase), thus enhancing the local potency and thus the beneficial effects of 25-hydroxy Vitamin D and 1,25 dihydroxy D (and allowing one to reduce the dosage of Vitamin D in a product with a progestin as compared to a product not including a progestin, if desired). Furthermore, it is believed that the combination of a progestin with 25-hydroxy Vitamin D compounds provides an even more pronounced biologic effect because of the synergistic interaction. The combination of a progestin with a 25-hydroxy Vitamin D compound provides a potent way to target vitamin D effects in the ovarian epithelium, in that vitamin D activity can be locally enhanced in the ovarian epithelium, without the potential harmful effects of high dosages of vitamin D administered systemically to achieve the same localized effect in the ovary. It is contemplated that the same beneficial effects would extend beyond the ovary to other organ sites including the breast, colon, immune system, prostate and cardiovascular system. Finally, it is also believed that the higher dosages of a 25-hydroxy Vitamin D compound will suppress the parathyroid hormone, and thereby minimize release of calcium from bones and improving bone density.

Embodiments of the invention include a composition containing a 25-hydroxy Vitamin D compound combined with a progestin. Higher dosages of 25-hydroxy Vitamin D compounds are contemplated as set forth herein, especially for products adapted for less than daily administration of the 25-hydroxy Vitamin D compound.

Embodiments further include 25-hydroxy Vitamin D compounds in oral contraceptive products (“OC”) and hormone replacement therapy (“HRT”) products containing progestin. These products of this embodiment of the invention include multiple sequential daily dosages, typically adapted for a cycle of 28 days (although longer and shorter cycles are within the scope of the invention). These products further include a 25-hydroxy Vitamin D compound.

In one embodiment, the range of dosages of 25-hydroxy Vitamin D is 17.5 to 280 mcg per week, with 87.5, 140, 175 and 280 mcg being some preferred dosages. Preferred dosages include at least 41 mcg per day of 25-hydroxy Vitamin D. According to the present invention, one could administer the weekly dosage of Vitamin D on one day per week, or alternatively on in divided doses on two or more consecutive days, or on two non-adjacent days with each day having one-half of the weekly amount. In other embodiments, the 25-hydroxy Vitamin D3 could be dosed more frequently, such as every 3, 4, 5 or 6 days, or twice a week. The dosages would be adjusted from those expressed above in this paragraph to account for the more frequent dosing. In another embodiment, the Vitamin D is dosed every two weeks or less frequently, for example, 35-560 mcg once every two weeks. Those dosages could be administered in two or three or more consecutive days every two weeks. For example, one could administer 25-hydroxy Vitamin D3 on days 6 and 7 and then again on days 20 and 21 of a four week cycle, with each of the dosages on those four days being at least 17.5 mcg 25-hydroxy Vitamin D3, and more preferably 35, 70 or 105 mcg.

Alternatively, the product could be adapted for administration of the 25-hydroxy Vitamin D compound once per cycle (e.g., once every four weeks). Preferred dosages would include at least 140 mcg every four weeks, and more preferably 280 mcg, 350 mcg or 420 mcg every four weeks. The 25-hydroxy Vitamin D compound can be administered over several days at one point in the month (or every four weeks). For example, the 25-hydroxy Vitamin D compound could be adapted to be administered in an oral contraceptive pharmaceutical combination over one 7-day period. Preferably the Vitamin D would be provided when the level of progestin is highest in the cycle. This could occur as the progestin dosage is pulsed at higher dosages on one or more days of each 28 day cycle. For example, in a triphasic OC regimen, the 25-hydroxy Vitamin D3 could be administered during all seven days when the progestin dosage is the highest (if other Vitamin D compounds are used, such as regular Vitamin D or calcitriol, they would be preferably administered during the day or days of highest progestin and/or estrogen dosages according to one aspect of the present invention). However, the 25-hydroxy Vitamin D could be administered during other 7-day periods (including the placebo days), or a shorter number of days of the cycle. If administered during a 7-day period in a 28-day cycle, the preferred dosages would include at least 12.5 mcg on each of the 7 days, and more preferably 12.5-60 mcg on each of the days, with 20, 25, 40 and 60 mcg being some preferred dosages for each of the days.

For HRT products containing both estrogen and progestin, another aspect of the invention involves administering the 25-hydroxy Vitamin D compound on a non-daily basis by providing it only when the progestin is administered. In some HRT products, estrogen alone is administered for 14 days, followed by 14 days of estrogen and progestin. It is believed that the 25-hydroxy Vitamin D compound is preferably administered on the same days as the progestin. If administered on all 14 days, the dosage of 25-hydroxy Vitamin D would preferably be at least 2.5 mcg on each day, and more preferably 2.5-30 mcg for each day, with 5, 10, 20 and 30 mcg being some preferred daily dosages.

In another alternative of the invention, the product is adapted to provide a 25-hydroxy Vitamin D compound with one or more other Vitamin D compounds. For example, one could provide 25-hydroxy Vitamin D3 in accordance the schedules above, but also provide Vitamin D3 or calcitriol. In one embodiment, the product is adapted to provide 25-hydroxy Vitamin D3 and calcitriol in the same composition for administration on the same day. In another embodiment, the product is adapted for the two Vitamin D compounds to be administered on different days. In these embodiments of the invention, the dosage of 25-hydroxy Vitamin D could be lower (e.g., one-half of the above dosages).

The products are preferably provided in a manner to enhance compliance. For example, the product could include a pill pack for the cycle. The 25-hydroxy Vitamin D3 could be included in the same pills containing the estrogen and/or progestin on specified days. Alternatively, the 25-hydroxy Vitamin D3 could be in separate pills set in the pill pack in a manner indicating or suggesting that they are for administration on the same day as hormone-containing pills or on days where hormones are not administered for specified days.

### BRIEF DESCRIPTION OF DRAWINGS

FIG. 1 shows the effect of 1,25 (OH)₂Vitamin D₃ and progesterone on cell viability (OVCAR 3 cells) in an MTS assay. The figure shows the data for percent viability for untreated cells, cells treated with 1,25 (OH)₂Vitamin D₃, cells treated with progesterone, and cells treated with a combination of 1,25 (OH)₂Vitamin D₃ and progesterone.

FIG. 2 shows the effect of 1,25 (OH)₂Vitamin D₃ and progesterone on cell viability (OVCAR 5 cells) in an MTS assay. The figure shows the data for percent viability for untreated cells, cells treated with 1,25 (OH)₂Vitamin D₃, cells treated with progesterone, and cells treated with a combination of 1,25 (OH)₂Vitamin D₃ and progesterone.

FIG. 3 shows the effect of 1,25 (OH)₂Vitamin D₃ and progesterone on the viability of immortalized human ovarian epithelial cells (HIO-118V) in an MTS assay. The figure shows the data for percent viability for untreated cells, cells treated with 1,25 (OH)₂Vitamin D₃, cells treated with three levels of progesterone, and cells treated with three combinations of 1,25 (OH)₂Vitamin D₃ and progesterone.

FIG. 4 shows the effect of 1,25 (OH)₂Vitamin D₃ and progesterone on apoptosis in OVCAR3 cells via the TUNEL method at 24 hours and at 48 hours. The Figure shows data for untreated cells, two levels of progesterone treated cells, one level of 1,25 (OH)₂Vitamin D₃ treated cells, and two levels of the combination treatment.

FIG. 5 shows the demonstration of induction of apoptosis by 1,25 (OH)₂ Vitamin D₃ and progesterone on OVCAR3 cells via the TUNEL method using flow cytometry. FIG. 5 shows untreated cells, cells treated with progesterone treated, cells treated with 1,25 (OH)₂Vitamin D₃ and cells treated with the combination of the two.

FIG. 6 shows the effects of the progesterone, 1,25 (OH)₂Vitamin D₃ and genistein on the expression of the Vitamin D 24 hydroxylase enzyme on OVCAR 3 cells by western blot.

FIG. 7A shows the effect of 1,25 (OH)₂Vitamin D₃, and genistein on cell viability (OVCAR 3 cells) in an MTS assay. The figure shows the data for percent viability for untreated cells, cells treated with 1,25 (OH)₂Vitamin D₃, cells treated with genistein, and cells treated with combinations of 1,25 (OH)₂Vitamin D₃ and genistein.

FIG. 7B shows the effect of 1,25 (OH)₂Vitamin D₃, and genistein on cell viability (OVCAR 5 cells) in an MTS assay. The figure shows the data for percent viability for untreated cells, cells treated with 1,25 (OH)₂Vitamin D₃, cells treated with genistein, and cells treated with combinations of 1,25 (OH)₂Vitamin D₃ and genistein.

FIG. 7C shows the effect of 1,25 (OH)₂Vitamin D₃ and genistein on cell viability (immortalized human ovarian epithelial cells (HIO-118V)) in an MTS assay. The figure shows the data for percent viability for untreated cells, cells treated with 1,25 (OH)₂Vitamin D₃, cells treated with genistein, and cells treated with combinations of 1,25 (OH)₂Vitamin D₃ and genistein.

FIG. 8A shows the effect of 1,25 (OH)₂Vitamin D₃, genistein and progesterone on cell viability (OVCAR 3 cells) in an MTS assay. The figure shows the data for percent viability for untreated cells, cells treated with 1,25 (OH)₂Vitamin D₃, cells treated with genistein, cells treated with progesterone, and cells treated with combinations thereof.

FIG. 8B shows the effect of 1,25 (OH)₂Vitamin D₃, genistein and progesterone on cell viability (OVCAR 5 cells) in an MTS assay. The figure shows the data for percent viability for untreated cells, cells treated with 1,25 (OH)₂Vitamin D₃, cells treated with genistein, cells treated with progesterone, and cells treated with combinations thereof.

FIG. 8C shows the effect of 1,25 (OH)₂Vitamin D₃, genistein and progesterone on cell viability (immortalized human ovarian epithelial cells (HIO-118V)) in an MTS assay. The figure shows the data for percent viability for untreated cells, cells treated with 1,25 (OH)₂Vitamin D₃, cells treated with genistein, cells treated with progesterone, and cells treated with combinations thereof.

### DETAILED DESCRIPTION OF THE INVENTION

The inventor has discovered that the combination of vitamin D with progestins causes synergistic inhibition of cell viability in cells derived from the human ovarian epithelium. Furthermore, the inventor has discovered that progestin causes inhibition and or degradation of 24 hydroxylase, the enzyme that inactivates 1,25-hydroxy Vitamin D, and other 25 hydroxylated Vitamin D compounds. It is thus contemplated that adding a progestin to vitamin D will prolong the local half life of 1,25-hydroxy Vitamin D and 25 D in the ovarian epithelium (via inhibition/degradation of 24 hydroxylase), thus enhancing 1,25-dihydroxy Vitamin D's and 25-hydroxy Vitamin D's local potency, as well as enhancing local production of 1,25 dihydroxy D from higher local levels of 25 hydroxy D, and thus greatly enhancing the ovarian cancer preventive effect of Vitamin D. For this invention, the 25-hydroxy form of vitamin D would be preferred because higher peak local levels of 25-hydroxy Vitamin D (achieved via administration of 25-hydroxy Vitamin D) would achieve higher local conversion to and thus local levels of the active hormone 1,25-hydroxy Vitamin D in the ovarian epithelium. Moreover, because less 25-hydroxy Vitamin D would be degraded (via inhibition of the 24 hydroxylase enzyme by progestin), then it is anticipated that even higher levels of 25-hydroxy Vitamin D would be achieved, having an even more enhanced and pronounced effect on local production of 1,25-hydroxy Vitamin D. Finally, via inhibition of 1,25-hydroxy Vitamin D degradation (again via inhibition of 24 hydroxylase by progestin), the hormone progestin when combined with 25-hydroxy Vitamin D would achieve the maximum local potency of vitamin D, thereby achieving a maximum beneficial effect of the vitamin, and maximum protection against neoplastic transformation of the ovarian surface epithelium. In the alternative to obtaining the higher effective dosage of Vitamin D, one could reduce the dosage of Vitamin D in a product with a progestin as compared to a product not including a progestin, if desired, to obtain the same Vitamin D benefits.

The combination of a progestin with 25-hydroxy Vitamin D provides a potent way to target vitamin D effects in the ovarian epithelium and other organ sites, in that 25-hydroxy Vitamin D activity can be locally enhanced in the ovarian surface epithelium or other organ sites, without the potential harmful effects of high dosages of 1,25 (OH)2 D3 administered systemically to achieve the same localized effect in the ovary. Finally, it is believed that the higher dosages of 25-hydroxy Vitamin D will suppress the parathyroid hormone, and thereby minimizing release of calcium from bones and improving bone density.

Applicant has shown that progesterone causes inhibition of cell viability in cells derived from the human ovarian epithelium when combined with 1,25 dihydroxy Vitamin D. Further, progesterone causes both degradation and decreases production of 24 hydroxylase in the ovcar-3 ovarian cancer cell line. Progesterone and vitamin D combined had the most marked impact on cell viability. Isobolographic analysis of the data demonstrates that the combination of progesterone with vitamin D confers synergistic effects on cell death/cell viability. It is believed that beneficial effects would occur with other progestins. It is also contemplated that beneficial effects would extend beyond the beneficial effects on the ovary to beneficial effects at other organ sites including the breast, colon, immune system and cardiovascular system. It is known that genistein causes decreased production of 24 hydroxylase. The combination of progesterone and genistein is even more potent, causing even more decrease in 24 hydroxylase. Also, the combination of progestin, vitamin D and Genistein is even more potent, conferring the greatest impact on cell viability.

The present invention generally relates to products and methods combining hormonal products with 25-hydroxy Vitamin D3 at particular dosages and schedules to confer enhanced skeletal and non skeletal benefits, including reducing the risk of certain cancers. The administration of 25-hydroxy Vitamin D3 with the other hormones is believed to maximally enhance benefits.

Embodiments of the invention include compositions containing 25-hydroxy Vitamin D combined with a progestin. Embodiments include 17.5-400 mcg of 25-hydroxy Vitamin D combined with a progestin and/or an estrogen, with 17.5-40 mcg, 41-100 mcg, 41-200 mcg, 25-40 mcg, 17.5-35 mcg, 40-70 mcg, 87.5-140 mcg, and 87.5-280 mcg of 25-hydroxy Vitamin D being some preferred dosage ranges. Preferred dosages include 17.5 mcg, 20 mcg, 25 mcg, 28 mcg, 30 mcg, 35 mcg, 40 mcg, 41 mcg, 44 mcg, 56 mcg, 88 mcg, 140 mcg, 175 mcg, 280 mcg, 350 mcg and 400 mcg, with the invention including the use of dosages in each of the separate ranges between Such specific dosages and the invention including combining such 25-hydroxy Vitamin D dosages with a progestin. Preferred dosages include at least 41 mcg per day of 25-hydroxy Vitamin D.

Embodiments further include 25-hydroxy Vitamin D in oral contraceptive products (“OC”) and hormone replacement therapy (“HRT”) products containing estrogen and/or progestin. Any of the known OC and HRT regimens can be adapted to include 25-hydroxy Vitamin D in accordance with the present invention. These products of this embodiment of the invention include multiple sequential daily dosages, typically adapted for a cycle of 28 days (although longer and shorter cycles are within the scope of the invention). These products further include 25-hydroxy Vitamin D. In one embodiment, the product is adapted for administration of the 25-hydroxy Vitamin D on a daily basis. The daily dosages of 25-hydroxy Vitamin D include 25-80 mcg of 25-hydroxy Vitamin D, with 25-40 mcg being preferred, and with preferred dosages 17.5 mcg, 25 mcg, 30 mcg, 35 mcg, 40 mcg, 60 mcg and 80 mcg and with the invention including the use of dosages in each of the separate ranges between such specific dosages. The higher dosages of 25-hydroxy Vitamin D are especially preferred for some embodiments and 25-hydroxy Vitamin D3 is especially preferred.

In other embodiments, the product is adapted for administration of 25-hydroxy Vitamin D3 on basis of less frequently than daily, including with OC and HRT regimens. In preferred embodiments, the range of dosages of 25-hydroxy Vitamin D3 is 17.5 to 280 mcg per week, with 44-175 mcg, 28-140 mcg, 35-87.5 mcg, 44-87.5 mcg, 56-140 mcg, 56-140 mcg and 87.5-175 mcg of 25-hydroxy Vitamin D being preferred dosage ranges and with preferred weekly dosages including 17.5 mcg, 28 mcg, 35 mcg, 40 mcg, 41 mcg, 44 mcg, 56 mcg, 70 mcg, 88 mcg, 140 mcg, 175 mcg, 280 mcg, with the invention including the use of dosages in each of the separate ranges between such specific dosages. The higher dosages of 25-hydroxy Vitamin D are especially preferred for some embodiments and 25-hydroxy Vitamin D3 is especially preferred. According to the present invention, one could administer the weekly dosage of Vitamin D one day per week, or alternatively on two or more consecutive days, or on two non-adjacent days with each day having one-half of the weekly amount. In other embodiments, the 25-hydroxy Vitamin D could be dosed every two weeks or less frequently, for example, at least 35 mcg, or preferably 175 mcg once every two weeks. Those dosages could be administered in two or three or more consecutive days every two weeks. For example, one could administer 25-hydroxy Vitamin D3 on days 6 and 7 and then again on days 20 and 21 of a four week cycle, with each of the dosages on those four days being at least 17.5 mcg 25-hydroxy Vitamin D3, and more preferably 35, 70 or 105 mcg, with the invention including the use of dosages in each of the separate ranges between such specific dosages. Preferably, with any OC and HRT regimens, the 25-hydroxy Vitamin D is dosed when the amount of progestin and/or estrogen is the highest or on the day-after administration of the highest amount of progestin and/or estrogen.

Alternatively, the product could be adapted for administration of the 25-hydroxy Vitamin D3 once per cycle (e.g., once every four weeks). Preferred dosages would include at least 140 mcg every four weeks, and more preferably 280 mcg, 350 mcg, or 420 mcg every four weeks. Higher dosages of up to 700 mcg could be used in dosed once a month or every four weeks, or even less frequently (once every 90 days in one day or over 2-7 consecutive days in the cycle). The 25-hydroxy Vitamin D3 can be administered over several days at one point in the month (or every four weeks). For example, the 25-hydroxy Vitamin D3 could be adapted to be administered in an oral contraceptive pharmaceutical combination over one 7-day period. Preferably the Vitamin D would be provided when the level of progestin and/or estrogen is highest in the cycle. For example, in a triphasic OC regimen, the 25-hydroxy Vitamin D3 could be administered during all seven days when the progestin dosage is the highest (if other Vitamin D compounds are used, such as regular Vitamin D or calcitriol, they would be preferably administered during the day or days of highest progestin according to one aspect of the present invention). However, the 25-hydroxy Vitamin D could be administered during other 7-day periods (including the placebo days), or a shorter number of day(or less) of the. If administered during a 7-day period in a 28-day cycle, the preferred dosages would include, at least 2.5 mcg on each of the 7 days, with 4, 5 and 10 mcg being alternative dosages. More preferably one would administer 12.5-60 mcg on each of the 7 days, with 20, 25, 40 and 60 mcg being some preferred dosages for each of the days, with the invention including the use of dosages in each of the separate ranges between such specific dosages.

For HRT products containing both estrogen and progestin, another aspect of the invention involves administering the 25-hydroxy Vitamin D3 on a non-daily basis by providing it only when the progestin is administered. In some HRT products, estrogen alone is administered for 14 days, followed by 14 days of estrogen and progestin. It is believed that the 25-hydroxy Vitamin D3 is preferably administered on the same days as the progestin. If administered on all 14 days, the dosage of 25-hydroxy Vitamin D3 would preferably be at least 2.5 mcg on each day, and more preferably 2.5-30 mcg for each day, with 5, 10, 20 and 30 mcg being some preferred daily dosages, with the invention including the use of dosages in each of the separate ranges between such specific dosages. Another HRT product has 3 days on progestin followed by 3 days off progestin. In one aspect of the invention, one would adapt the product to administer Vitamin D only when progestin is administered. The Vitamin D could be at any of the dosages discussed herein. For example, one could administer the dosages mentioned herein for products adapted for admistration of Vitamin D every day, but double the dosages to reflect that the Vitamin D is administered only every 50% of the days.

In another alternative of the invention, the product is adapted to provide 25-hydroxy Vitamin D3 with one or more other Vitamin D compounds. For example, one could provide 25-hydroxy Vitamin D3 in accordance the schedules above, but also provide Vitamin D3 or calcitriol, preferably on days when 25-hydroxy Vitamin D3 is not provided. In these embodiments of the invention, the dosage of 25-hydroxy Vitamin D3 could be lower (e.g., one-half of the above dosages).

The products are preferably provided in a manner to enhance compliance. For example, the product could include a pill pack for the cycle. The 25-hydroxy Vitamin D3 could be included in the same pills containing the estrogen and/or progestin on specified days. Alternatively, the 25-hydroxy Vitamin D3 could be in separate pills set in the pill pack in a manner indicating or suggesting that they are for administration on the same day as hormone-containing pills or on days where hormones are not administered for specified days.

Various combinations of progestin and estrogen that have been used in OCs and HRTs are shown in Tables 1, 2 and 3. The present invention includes adapting these products to include 25-hydroxy Vitamin D in the dosages described herein. The 25-hydroxy Vitamin D can be included on a daily basis, or less frequently than daily. For any bi-phasic or tri-phasic regimens, if the 25-hydroxy Vitamin D is to be administered on a less frequently than daily basis, it is preferred that the 25-hydroxy Vitamin D is administered when the progestin is highest.

TABLE 1
Combinations of Progestin and Estrogen in OCs

| | Dose | | Dose |
|---|---|---|---|
| Progestin | (mg) | Estrogen | (mg) |
| Norethynodrel | 9.85 | Mestranol | 0.150 |
| | 5.00 | | 0.075 |
| | 2.50 | | 0.036 |
| | 2.50 | | 0.100 |
| Norethindrone | 10.00 | Mestranol | 0.060 |
| | 2.00 | | 0.100 |
| | 1.00 | | 0.050 |
| | 1.00 | | 0.080 |
| Norethindrone | 1.00 | Ethinyl | 0.050 |
| | 0.50 | estradiol (EE) | 0.035 |
| | 0.40 | | 0.035 |
| Norethindrone | 2.50 | EE | 0.050 |
| Acetate | 1.00 | | 0.050 |
| | 0.60 | | 0.030 |
| | 1.50 | | 0.030 |
| | 1.00 | | 0.020 |
| Ethynodiol | 1.00 | Mestranol | 0.100 |
| Diacetate | | | |
| Ethynodiol | 1.00 | EE | 0.050 |
| Diacetate | | | |
| dl-Norgestrel | 0.50 | EE | 0.050 |
| | 0.30 | | 0.030 |

Equivalencies
50 mg Mestranol = 35 mg Ethinyl estradiol (EE)
0.5 mg dl-Norgestrel = 2 mg Norethindrone

Table 2 below lists the progestin and estrogen content of some commercial regimens.

TABLE 2
Composition of Selected Currently Marketed Oral Contraceptives

| | |
|---|---|
| μg | Mg |

COMBINATION TYPE

| | | | | |
|---|---|---|---|---|
| FIXED TYPE | | | | |
| Estrogen content = 50 μg: | | | | |
| Ortho-Novum 1/50 | Mestranol | 50 | Norethindrone | 1.0 |
| Norinyl 1/50 | Mestranol | 50 | Norethindrone | 1.0 |
| Ovcon 50 | Ethinyl estradiol | 50 | Norethindrone | 1.0 |
| Ovral | Ethinyl estradiol | 50 | Norgestrel | 0.5 |
| Norlestrin 2.5/50 | Ethinyl estradiol | 50 | Norethindrone acetate | 2.5 |
| Norlestrin 1/50 | Ethinyl estradiol | 50 | Norethindrone acetate | 1.0 |
| Demulen | Ethinyl estradiol | 50 | Ethynodiol | 1.0 |
| Zovia | Ethinyl estradiol | 50 | Ethynodiol diacetate | 1.0 |
| Estrogen content = 35 μg: | | | | |
| Ortho-Novum 1/35 | Ethinyl estradiol | 35 | Norethindrone | 1.0 |
| Norinyl 1 + 35 | Ethinyl estradiol | 35 | Norethindrone | 1.0 |
| Modicon | Ethinyl estradiol | 35 | Norethindrone | 0.5 |
| Brevicon | Ethinyl estradiol | 35 | Norethindrone | 0.5 |
| Ovcon 35 | Ethinyl estradiol | 35 | Norethindrone | 0.4 |
| Demulen 1/35 | Ethinyl estradiol | 35 | Ethynodiol diacetate | 1.0 |
| Ortho-cyclen | Ethinyl estradiol | 35 | Norgestimate | 0.25 |
| Necon | Ethinyl estradiol | 35 | Norethindrone | 1 |
| Norethin | Ethinyl estradiol | 35 | Norethindrone | 1 |
| Ovcon | Ethinyl estradiol | 35 | Norethindrone | 0.4 |
| Tri-Norinyl | Ethinyl estradiol | 35 | Norethindrone | 0.5 |
| Zovia | Ethinyl estradiol | 35 | Ethynodiol diacetate | 1 |
| Estrogen content = 30 μg: | | | | |
| Loestrin 1.5/30 | Ethinyl estradiol | 30 | Norethindrone acetate | 1.5 |
| Nordette | Ethinyl estradiol | 30 | Levonorgestrel | 0.15 |
| Lo-Ovral | Ethinyl estradiol | 30 | Norgestrel | 0.3 |
| Desogen | Ethinyl estradiol | 30 | Desogestrel | 0.15 |
| Ortho-cept | Ethinyl estradiol | 30 | Desogestrel | 0.15 |
| LevLen | Ethinyl estradiol | 30 | Desogestrel | 0.15 |
| Levora | Ethinyl estradiol | 30 | Levonorgestrel | 0.15 |
| Minulet | Ethinyl estradiol | 30 | Gestrodene | 0.075 |
| Eugynon 30 | Ethinyl estradiol | 30 | Levonorgestrel | 0.250 |
| Seasonale | Ethinyl estradiol | 30 | Levonorgestrel | 0.15 |
| (84 days/7 days placebo) | | | | |
| Yasmin | Ethinyl estradiol | 30 | Drospirenone | 3.00 |
| Estrogen content = 20 μg: | | | | |
| Loestrin 1/20 | Ethinyl estradiol | 20 | Norethindrone acetate | 1.0 |
| Alesse | Ethinyl estradiol | 20 | Levonorgestrel | 0.1 |
| LevLite | Ethinyl estradiol | 20 | Levonorgestrel | 0.1 |
| Mircette | Ethinyl estradiol | 20 | Desogestrel | 0.15 |
| Femodette | Ethinyl estradiol | 20 | Gestodene | .075 |
| Yaz | Ethinyl estradiol | 20 | Drospirenone | 3.00 |
| BIPHASIC TYPE | | | | |
| Ortho-Novum 10/11 | | | | |
| First 10 days | Ethinyl estradiol | 35 | Norethindrone | 0.5 |
| Next 11 days | Ethinyl estradiol | 35 | Norethindrone | 1.0 |
| Mircette | | | | |
| First 21 days | Ethinyl estradiol | 20 | Desogestrel | 0.15 |
| Next 5 days | Ethinyl estradiol | 10 | Desogestrel | 0 |
| Next 2 days (Placebo) | | | | |
| BiNovum | | | | |
| First 7 days | Ethinyl estradiol | 35 | Norethindrone | 0.5 |
| Next 14 days | Ethinyl estradiol | 35 | Norethindrone | 1.0 |
| Seasonique | | | | |
| First 84 days | Ethinyl estradiol | 30 | Levonorgestrel | 0.15 |
| Next 7 days | Ethinyl estradiol | 10 | | |
| Jenest 28 | | | | |
| First 7 days | Ethinyl estradiol | 35 | Norethindrone | 0.5 |
| Next 14 days | Ethinyl estradiol | 35 | Norethindrone | 1.0 |
| TRIPHASIC TYPE | | | | |
| Ortho-Novum 7/7/7 | | | | |
| First 7 days | Ethinyl estradiol | 35 | Norethindrone | 0.5 |
| Second 7 days | Ethinyl estradiol | 35 | Norethindrone | 0.75 |
| Third 7 days | Ethinyl estradiol | 35 | Norethindrone | 1.0 |
| Tri-Norinyl | | | | |
| First 7 days | Ethinyl estradiol | 35 | Norethindrone | 0.5 |
| Next 9 days | Ethinyl estradiol | 35 | Norethindrone | 1.0 |
| Next 5 days | Ethinyl estradiol | 35 | Norethindrone | 0.5 |
| Triphasil | | | | |
| First 6 days | Ethinyl estradiol | 30 | Levonorgestrel | 0.05 |
| Second 5 days | Ethinyl estradiol | 40 | Levonorgestrel | 0.075 |
| Third 10 days | Ethinyl estradiol | 30 | Levonorgestrel | 0.125 |
| Tri-LevLen | | | | |
| First 6 days | Ethinyl estradiol | 30 | Levonorgestrel | 0.05 |
| Second 5 days | Ethinyl estradiol | 40 | Levonorgestrel | 0.075 |
| Third 10 days | Ethinyl estradiol | 30 | Levonorgestrel | 0.125 |
| Ortho Tri-Cyclen | | | | |
| First 7 days | Ethinyl estradiol | 35 | Norgestimate | 0.18 |
| Second 7 days | Ethinyl estradiol | 35 | Norgestimate | 0.215 |
| Third 7 days | Ethinyl estradiol | 35 | Norgestimate | 0.25 |
| Ortho Tri-Cyclen Lo | | | | |
| First 7 days | Ethinyl estradiol | 25 | Norgestimate | 0.18 |
| Second 7 days | Ethinyl estradiol | 25 | Norgestimate | 0.215 |
| Third 7 days | Ethinyl estradiol | 25 | Norgestimate | 0.25 |
| Cyclessa | | | | |
| First 7 days | Ethinyl estradiol | 25 | Desogestrel | 0.10 |
| Second 7 days | Ethinyl estradiol | 25 | Desogestrel | 0.125 |
| Third 7 days | Ethinyl estradiol | 25 | Desogestrel | 0.15 |
| Tri-Minulet | | | | |
| First 6 days | Ethinyl estradiol | 30 | Gestodene | 0.05 |
| Next 5 days | Ethinyl estradiol | 40 | Gestodene | 0.7 |
| Next 10 days | Ethinyl estradiol | 30 | Gestodene | 0.10 |
| Estrostep | | | | |
| First 5 days | Ethinyl estradiol | 20 | Norethindrone | 1.0 |
| Next 7 days | Ethinyl estradiol | 30 | Norethindrone | 1.0 |
| Next 9 days | Ethinyl estradiol | 35 | Norethindrone | 1.0 |

PROGESTOGEN ONLY

| | | | |
|---|---|---|---|
| Micronor | None | Norethindrone | 0.35 |
| Nor Q.D. | None | Norethindrone | 0.35 |
| Ovrette | None | Norgestrel | 0.075 |
| Cerazette | None | Desogestrel | 0.075 |
| Femulen | None | Ethynodiol diacetate | 0.50 |
| Microval | None | Levonorgetrel | 0.03 |

Table 3 lists the estrogen contents (and other hormonal ingredients, where applicable) for various hormone replacement products.

TABLE 3
Content of Common Hormone Replacement Regimens

| Name | Estrogen | mg | Other Hormone | Mg | Regimen |
|---|---|---|---|---|---|
| Premarin | Conjugated Estrogens | 0.3/ | — | — | 0.3-1.25 mg daily, |
| (tablet) | | 0.625/ | | | administered continuously |
| | | 0.9/ | | | (daily, with no breaks) or |
| | | 1.25 | | | days 1-25 of month |
| | | 2.5/ | | | |
| Premarin | Conjugated Estrogens | 0.625/1 g | — | — | ½-2 g daily; 3 weeks on, 1 |
| (cream) | | of cream | | | week off |
| Prempro | Conjugated Estrogens | 0.625 | Medroxy- | 5 mg | Continuous |
| (tablets) | | | progesterone acetate | or 2.5 mg | |
| Premphase | Conjugated Estrogens | 0.625 | Medroxy- | 5 mg | Continuous |
| (two types | | | progesterone acetate | | |
| of tablets) | | | | | |
| Days 1-14 | Yes | | No | | |
| Days 15-28 | Yes | | Yes | | |
| Estratest | Esterified Estrogens | 1.25 | Methyl-testosterone | 2.5 mg | 3 weeks on; 1 week off |
| | | | (Androgen) | | |
| Estratest H.S. | Esterified Estrogens | 0.625 | Methyl-testosterone | 1.25 mg | 3 weeks on; 1 week off |
| | | | (Androgen) | | |
| Estrace | Estradiol | .5-2 | — | — | 3 weeks on; 1 week off |
| (tablets) | | | | | |
| Climara | Estradiol | 0.025/ | — | — | Continuous |
| (patch) | (four different patches; | 0.05/ | | | |
| | dosages per day) | 0.075/ | | | |
| | | 0.10 mg | | | |
| | | released | | | |
| | | per day | | | |

TABLE 4
Progestins Classification, and Recommended Doses for Endometrial Protection
in Hormonal Replacement Therapy (when used with various estrogens, sequential
administration of progestins 10-14 days per month, of estrogen therapy)

| | | | Recommended |
|---|---|---|---|
| | | Dose/oral | dose (mg) |
| | | tablet | for endometrial |
| Progestin type | | (mg) | protection (α) |
| 1. | PREGNANES | | |
| 1.1 | PROGESTERONE (P) | | |
| | Micronized P | 100 | 200-300 |
| | Vaginal progesterone (cream) | 45 or 90 | 45 |
| | retro-progesterone (didrogesterone) | 5 or 10 | 20 |
| 1.2 | 17 HYDROXY PROGESTERONES | | |
| | Chlormadinone acetate | 2, 5 | 10 |
| | Medroxyprogesterone acetate | 2.5, 5, 10 (seq) | 10 (seq) |
| | | 2.5 cc with CEE | 2.5 (cc) |
| | Cyproterone acetate | 1 (with E2V 2 mg) | 1 |
| | | 50 | |
| 1.3 | 19-NORPROGESTERONES | | |
| | Promegestone (R5020) | 0.125, 0.250 | 0.25-0.5 |
| | Demegestone | 0.5 | 1 |
| | Nomegestrol Acetate | 5 | 5-10 |
| | Trimegestone | 0.25, 0.5 | 0.5 |
| | Nestorone (CVR or TTS) | 0.05, 0.075, 0.1 | 0.005-0.1 |
| | Medrogestone | 5 | 5-10 |
| 2. | TESTOSTERONE DERIVATIVES | | |
| 2.1 | ESTRANES | | |
| | Norethisterone | 0.35, 5 | 1 |
| | Norethisterone acetate | 0.5, 1.0 | 1 (oral); 0.25 (TTS) |
| | Norethindrone acetate | 0.5, 1.0 | |
| | Ethynodiol di-acetate | 2 | 2-4 |
| | Lynestrenol | 0.5, 5 | — |
| 2.2 | GONANES | | |
| | L-Norgestrel | 0.015, 0.075 | 0.15-0.5 |
| | Desogestrel | 0.5 or 2.0 | |
| | Norgestimate | 0.09 (3 days on, followed | |
| | | by 3 days off) with 1 mg | |
| | | 17B/E2 | |
| | Gestodene | 0.025-0.05 | 0.05 with E2 2 mg |
| | Dienogest (Hybrid progestin) | 2, 3, 4 | 3 and 4 |

Abbreviations:
CEE, conjugated estrogens;
E2, Estradiol;
E2V, Estradiol valerate;
CVR, contraceptive vaginal ring;
TTS, transdermal system;
P, progesterone;
cc, continuous combined;
seq, sequential.
Table 4 is reproduced from Progestins and Antiprogestins in Clinical Practice, with modifications, ed. by Sitruk-Ware (2000).

The OCs frequently come either with either 21 pills for a cycle or with 28 pills. The 28 pill products frequently include 7 days of placebo. As shown in the Tables above, there could be less than 7 days of placebo (e.g. Mircette). Other OCs have longer cycles (e.g., Seasonale). This invention would include the use of 25-hydroxy Vitamin D with each of the OCs and HRTs in Tables 1-4 above, as well as any other OCs and HRTs, at the normal dosages of progestin and estrogen. In one preferred embodiment, one uses the lowest level of estrogen and progestin. For products with only 21 pills, one could simply add 25-hydroxy Vitamin D for administration with one or more of the 21 pills, or one could modify the product to include dosages for all 28 days, with the 7 days of placebo having one or more days with 25-hydroxy Vitamin D. For any of the OC or HRT products mentioned herein, the 25-hydroxy Vitamin D can be administered every day or on a less frequent basis, at the dosages and frequencies described above.

Another OC is Lybrel (90 microgram levonorgestrel/20 microgram ethinyl estradiol tablets), a low dose, continuous, non-cyclic combination OC. HRT products can be administered every day. For such continuous OC or HRT products, the 25-hydroxy Vitamin D can be administered every day or on a less frequent basis, at the dosages and frequencies described above.

In addition, applicant disclosed various hormonal regimens in his disclosure of Ser. No. 09/798,453 filed Mar. 2, 2001, entitled Prevention Of Ovarian Cancer By Administration Of Products That Induce Biologic Effects In The Ovarian Epithelium. Applicant specifically incorporates herein by reference that entire disclosure. Any of the compositions and regimens of that prior disclosure can be altered in accordance with the present invention by the addition of a 25-hydroxy Vitamin D compound at the dosages and schedules described herein.

Applicant's invention is applicable to both mono-phasic and multi-phasic OC and HRT regimens as discussed herein where the dosage of progestin and/or estrogen is not altered from typical OC and/or HRT regimens. However, in another embodiment of the invention, the progestin dosages can be altered for one or more of the dosages in the cycle. If the progestin and/or estrogen dosage is to be altered in an embodiment, it is preferred that the progestin is increased, and that it is increased only for less than all of the cycle and preferably when the Vitamin D is administered. By the term “mono-phasic” as used herein, applicant means that within a cycle, the daily dosage of the therapeutically active estrogen and progestin compounds remains constant, except for placebo days, if any, in the regimen. For example, a regimen having 21 days of a constant level of progestin and estrogen with 7 days of placebo is mono-phasic as used herein. By the term “multi-phasic” as used herein, applicant means that within a cycle, the daily dosage of the therapeutically active estrogen and progestin compounds varies at least once so that there are at least two phases with different levels and/or types of therapeutically active compounds. Accordingly, as the “phase” term is used herein by applicant, each “phase” in a multi-phase regimen is either the first phase having one or more therapeutically active compounds or a subsequent phase having one or more therapeutically active compounds with different levels and/or types of therapeutically active compounds as compared to the immediately prior phase. For example, a 28-day regimen having 21 days of a constant level of progestin and estrogen with 7 days of a estrogen bridge would be multi-phasic as used herein, specifically bi-phasic. A regimen having 7 days of progestin at Dose amount A, followed by 7 days of progestin at dose amount B, followed by 7 days of progestin at dose amount A (at the same level as the first 7 days), followed by 7 days of placebo would be multi-phasic, having three phases and thus tri-phasic as those terms are used in this application.

This invention contemplates mono-phasic or multi-phasic OCP regimens containing a 25-hydroxy Vitamin D compound, with at least one or more of the daily dosages having a progestin compound in the range of 0.05-10 mg, with 0.075, 0.15, 0.25, 0.5, 0.8, 1.0, 1.2, 1.8, 2.5 and 5.0 mg being some preferred dosages, with the invention including ranges between those dosages. One aspect of the invention allows the use of lower amounts of progestin in some embodiments, as the vitamin D component enhances the benefit of the product, and thus less progestin could be necessary. The 25-hydroxy Vitamin D compound is provided at the preferred ranges and dosages described herein (preferably 25-hydroxy Vitamin D3).

The ratio of progestin to 25-hydroxy Vitamin D compound in a given dosage can vary. Some preferred ratios include 20:1, 15:1, 10:1, 5:1, 2.5:1, 1:1, 1:2.5 and 1:5 being some preferred progestin to 25-hydroxy Vitamin D compound ratios, with the invention including each of the separate ranges between those ratios, and the ratios varying based on the type and potency of progestin used, as well as the frequency of administration of the vitamin D compound. For products adapted for daily administration of the 25-hydroxy Vitamin D compound, the ratios are preferably in the range of 10:1 to 5:1, although other ratios are useful. For products adapted for less than daily administration of the 25-hydroxy Vitamin D compound, the ratios are preferably lower, in the range of 100:1 to 10:1, although other ratios are useful. These ratios are applicable to all progestins specified in this application, but are especially applicable progesterone and gonane progestins. For pregnane and estrane progestins, the preferred ratios can be 5-10 times higher than those specified earlier in this paragraph. For progesterone and Drospirenone, the amounts of progestin are higher as thus the ratoes are substantially higher.

In multi-phasic regimens, the 25-hydroxy Vitamin D compound is preferably provided when the progestin dosage is the highest. This daily dosage is administered at least one day, more preferable at least two days or alternatively at least 3 days. Preferred ranges for the length of this phase in multi-phasic regimens are from 1-15 days, from 2-11 days, and from 3-7 days. The estrogen level used in OC regimens preferably has no daily dosage exceeding 50 mcg EE dosage equivalent, and more preferably not to exceed 35 mcg, and some preferred dosages of 30 mcg and 25 mcg, even more preferably not to exceed 20 mcg, with 10 and 15 mcg being two other contemplated dosages, with EE being the preferred estrogen for OCs. Preferred ranges of the EE in the OC regimens are 10-35 mcg, 15-25 mcg, 20-35 mcg and 15-20 mcg. A weaker estrogen or an estrogen having antiestrogenic activity (Such as SERMs or phytoestrogens discussed below) can be used or added as a second estrogen to any of the regimens of this paragraph.

Alternatively, this invention contemplates a HRT regimen for post-menopausal women, and a HRT regimen for peri-menopausal women, having the ingredients and dosages mentioned above in this paragraph, except the estrogen dosages are 5 mcg or less EE dosage equivalent and again can substitute a SERM for EE or estradiol. The different estrogens that are used in HRT products include, for example: conjugated synthetic estrogens (e.g. Enjuvia) at daily dosages such as 0.3 mg, 0.45 mg, 0.625 mg and 1.25 mg; estrodiol acetate at daily dosages such as 0.45 mg, 0.9 mg and 1.8 mg; conjugated equine estrogens at daily dosages such as 0.3 mg, 0.45 mg, 0.625 mg, 0.9 mg and 1.25 mg; and 17-β estradiol at daily dosages Such as 0.5 mg, 1.0 mg and 2.0 mg. Daily dosages of ethynyl estradiol in HRT products include 5 mcg or less such as 2.5 mcg daily dosages. In HRT products using drospirenone, the daily amount of estrogen is typically 17-β estradiol at 1.0 mg per day. In HRT products using conjugated equine estrogens, low dose pills would include 0.3 or 0.45 mg per day of such estrogens, with the dosage of medroxy progesterone acetate at 1.5 mg on days when progestin is administered in such HRT regimens. Low dose EE HRT pills would include EE at 2.5 mcg per day, with norethinedrone acetate at 0.5 mg on the days when the progestin is administered.

Although lower dosages of progestins are preferred, it is contemplated that the higher doses of progestins can be used in another alternative for either monophasic, biphasic, triphasic or any other multi-phasic schedules can alternatively be given in units of time comprising 1 day, 1-3 days, 3-5 days, 6-10 days, 10-14 days, or longer. Furthermore, these units of time could be applicable to a regimen comprising a one month cycle, 2 month cycle, 3-6 month cycle or longer. It is further contemplated in one aspect of the invention that exemplary regimens according to this invention would consist of administering progestins in the lowest doses possible, except for the units of time during which Vitamin D is administered. The objective of this approach would be to devise a contraceptive regimen with the least side effects and least overall exposure to progestin, while at the same time maximizing benefits of the Vitamin D. An estrogen having a weak estrogenic activity or antiestrogenic activity can be added to any of the formulations mentioned in this paragraph in lieu of or in addition to the estrogen in the current formulation.

This invention contemplates mono-phasic or multi-phasic OCP or HRT regimens containing a 25-hydroxy Vitamin D compound, with at least one or more of the daily dosages having norgestimate in the range of 0.05-5.0 mg, including 0.09, 0.18, 0.215, and 0.25 mg, and in between such dosages, being some preferred dosages. For alternative embodiments having one or more dosages with increased progestin, the preferred dosages of norgestimate for such embodiments include at least one daily dosage 0.5, 0.8, or more preferably at least 1.2 mcg norgestimate, and even more preferably at least 1.8, and most preferably at least 2.5, with 1-7 such daily dosages being preferred, and 3-7 being more preferred. The remaining dosages can be, for example, the normal dosages found in the tables above.

This invention contemplates mono-phasic or multi-phasic OCP or HRT regimens containing a 25-hydroxy Vitamin D compound, with at least one or more of the daily dosages having 0.5-15 mg of one or more of the progestins from the group consisting of norethinedrone, and norethynodrel, preferably with dosages of 0.5 to 1.0 mg. For alternative embodiments having one or more dosages with increased progestin, the preferred dosages of norethindrone and norethynodrel for such embodiments include at least have higher dosages in at least one daily dosage, such as 2.1 mg, at least 2.5, more preferably at least 3.0, even more preferably at least 4.0, and up 5.0, with 1-7 such daily dosages being preferred, and 3-7 being more preferred. The remaining dosages can be, for example, the dosages found in the tables above.

This invention also contemplates mono-phasic or multi-phasic OCP or HRT regimens containing a 25-hydroxy Vitamin D compound, with at least one or more of the daily dosages having levonorgestrel in the range of 0.03-5.0 mg, with low dosages of 0.03, 0.05, 0.075, 0.090, 0.1. 0.125, 0.15, 0.20, 0.25 and 0.50 mg, and in between such dosages, being preferred. For alternative embodiments having one or more dosages with increased progestin, the preferred dosages of levonorgestrel for such embodiments include at least at least one daily dosage of levonorgestrel of at least 0.25, at least 0.5, at least 1.0, at least 1.5, or up to 2.0 mg, with 1-7 such daily dosages being preferred, and 3-7 being more preferred. The remaining dosages can be, for example, the dosages found in the tables above. One version of the invention of this paragraph is a mono-phasic regimen with 0.25 mg or more of levonorgestrel daily dosage with an estrogen daily dosage of less than 50 mcg EE dosage equivalent, and more preferably not to exceed 20 mcg, and most preferably not to exceed 15 mcg. Alternatively, a multi-phasic regimen is used with at least one phase having 0.25 mg or levonorgestrel or more and another phase having less levonorgestrel, preferably less than 0.25 mg levonorgestrel, with estrogen daily dosages of less than 50 mcg EE dosage equivalent, and more preferably not to exceed 20 mcg, and most preferably not to exceed 15 mcg.

This invention also contemplates mono-phasic or multi-phasic OCP or HRT regimens containing a 25-hydroxy Vitamin D compound, with at least one or more of the daily dosages having norgestrel in the range of 0.06-10 mg, with low dosages of 0.075, 0.15, 0.25, 0.3 and 0.5 mg and in between Such dosages being preferred. For alternative embodiments having one or more dosages with increased progestin, the preferred dosages of norgestrel for such embodiments include at least one daily dosage of norgestrel of at least 0.5 mg of norgestrel, alternatively at least 1.0, alternatively at least 1.5, and alternatively at least 2.0, and alternatively at least 3.0, or alternatively up to 4.0 mg, with 1-7 such daily dosages being preferred, and 3-7 being more preferred. The remaining dosages can be, for example, the dosages found in the tables above. One version of the invention of this paragraph is a mono-phasic regimen with 0.5 mg or more of norgestrel daily dosage with an estrogen daily dosage of less than 50 mcg EE dosage equivalent, and more preferably not to exceed 35 mcg, and even more preferably not to exceed 20 mcg, and most preferably not to exceed 15 mcg. Alternatively, a multi-phasic regimen is used with at least one phase having 0.5 mg or norgestrel or more and another phase having less norgestrel, preferably less than 0.5 mg norgestrel, with estrogen daily dosages of less than 50 mcg EE dosage equivalent, and more preferably not to exceed 35 mcg, even more preferably not to exceed 20 mcg, and most preferably not to exceed 15 mcg.

This invention also contemplates mono-phasic or multi-phasic OCP or HRT regimens containing a 25-hydroxy Vitamin D compound, with at least one or more of the daily dosages having norethindrone acetate in the range of 0.6-10 mg, with low dosages of 0.6, 1.0, 1.5 and 2.5 mg and in between such dosages being preferred. For alternative embodiments having one or more dosages with increased progestin, the embodiments include at least one daily dosage of preferably 1.0 mg of norethindrone acetate, alternatively at least 1.5, alternatively at least 2.0, and alternatively at least 2.5, and alternatively at least 3.0, or alternatively up to 4.0 mg, with 1-7 such daily dosages being preferred, and 3-7 being more preferred. The remaining dosages can be, for example, the dosages found in the tables above.

This invention also contemplates mono-phasic or multi-phasic OCP or HRT regimens containing a 25-hydroxy Vitamin D compound, with at least one or more of the daily dosages having desogestrel the range of 0.05-5.0 mg, with low dosages of 0.075, 0.10, 0.15, 0.3 and 0.50 mg and in between such dosages being preferred. For alternative embodiments having one or more dosages with increased progestin, the embodiments include at least at least one daily dosage of preferably at least 0.7 mg of desogestrel, alternatively at least 1.2, alternatively at least 1.8, and alternatively at least 2.4, and alternatively up to 3.0 mg, with 1-7 such daily dosages being preferred, and 3-7 being more preferred. The remaining dosages can be, for example, the dosages found in the tables above.

This invention also contemplates mono-phasic or multi-phasic OCP or HRT regimens containing a 25-hydroxy Vitamin D compound, with at least one or more of the daily dosages having dienogest or drospirenone in the range of 0.25-10 mg, with dosages of 0.5, 1.0, 1.5, 3 mg and 4.0 mg and in between such dosages being preferred. For alternative embodiments having one or more dosages with increased progestin, the embodiments include at least one daily dosage of dienogest or drospirenone of preferably at least 4.1 mg of dienogest or drospirenone, alternatively at least 5.0, alternatively at least 6.0, and alternatively at least 6.5, and alternatively at least 7.0, or alternatively up to 8.0, with 1-7 such daily dosages being preferred, and 3-7 being more preferred.

This invention also contemplates mono-phasic or multi-phasic OCP or HRT regimens containing a 25-hydroxy Vitamin D compound, with at least one or more of the daily dosages having progesterone in the range of 2-20 mg, with dosages of 4, 5, and 7.5 mg and in between such dosages being preferred. For alternative embodiments having one or more dosages with increased progestin, the embodiments include at least one daily dosage of progesterone of preferably at least 8 mg of progesterone, alternatively at least 10, alternatively up to at least 15 mg, or alternatively up to 15, with 1-7 such daily dosages being preferred, and 3-7 being more preferred.

This invention also contemplates mono-phasic or multi-phasic OCP or HRT regimens containing a 25-hydroxy Vitamin D compound, with at least one or more of the daily dosages having medroxyprogesterone acetate in the range of 1.0-40 mg, with low dosages of 1, 1.5, 2.5, 5.0 and 10 mg and in between such dosages being preferred. For alternative embodiments having one or more dosages with increased progestin, the embodiments include at least one phase with one or more of the daily dosages in one of the phases having at least 7 mg of medroxyprogesterone acetate, preferably at least 10, more preferably 12, and even more preferably 20, and most preferably 30 or more, with 1-7 Such daily dosages being preferred, and 3-7 being more preferred.

This invention also contemplates mono-phasic or multi-phasic OCP or HRT regimens containing a 25-hydroxy Vitamin D compound, with at least one or more of the daily dosages having ethynodiol diacetate in the range of 0.4-5 mg, with 0.5 to 1.5 mg being preferred. Other embodiments have at least one daily dosage having at least 2.0, and alternatively at least 3.0, or alternatively up to 4.0 mcg, with 1-7 such daily dosages being preferred, and 3-7 being more preferred. The remaining dosages can be, for example, the dosages found in the tables above. Alternatively, a multi-phasic regimen is used with at least one phase having 1.0 mg or ethinodiol diacetate or more and another phase having less ethinodiol diacetate, preferably less than 1.0 mg, with estrogen daily dosages of less than 50 mcg EE dosage equivalent, and more preferably not to exceed 35 mcg, even more preferably not to exceed 20 mcg, and most preferably not to exceed 15 mcg.

This invention also provides a method of contraception which comprises administering to a female of child bearing age for 23-25 consecutive days, a first phase combination of a progestin at a daily dosage of 40-500 mcg trimegestone, 250 mcg-4 mg dienogest, or 250 mcg-4 mg drospirenone, and an estrogen at a daily dosage equivalent in estrogenic activity to 10-30 mcg ethinyl estradiol for 9-13 days beginning on day 1 of the menstrual cycle, wherein the same dosage of the progestin and estrogen combination is administered in each of the 9-13 days, and a second phase combination of a progestin at a daily dosage of 40-500 mcg trimegestone, 250 mcg-4 mg dienogest, or 250 mcg-4 mg drospirenone, and an estrogen at a daily dosage equivalent in estrogenic activity to 10-30 mcg ethinyl estradiol, for 11-15 days beginning on the day immediately following the last day of administration of the first phase combination, wherein the same dosage of the progestin and estrogen combination is administered in each of the 11-15 days, provided that the daily dosage of second phase progestin is greater than the daily dosage of the first phase progestin and that the daily dosage of the second phase estrogen is greater than or equal to the daily dosage of the first phase estrogen and wherein the regimen is modified so that one or more of the daily dosages further includes a 25-hydroxy Vitamin D compound.

This invention further includes a method of contraception which comprises administering orally to a female of child bearing age for 23-25 consecutive days, a first phase combination of a progestin at a daily dosage selected from the group consisting of 40-500 mcg trimegestone, 250 mcg-4 mg dienogest, and 250 mcg-4 mg drospirenone, and an estrogen at a daily dosage equivalent in estrogenic activity to 10-30 mcg ethinyl estradiol for 3-8 days beginning on day 1 of the menstrual cycle, wherein the same dosage of the progestin and estrogen combination is administered in each of the 3-8 days, a second phase combination of a progestin at a daily dosage selected from the group consisting of 40-500 mcg trimegestone, 250 mcg-4 mg dienogest, and 250 mcg-4 mg drospirenone, and an estrogen at a daily dosage equivalent in estrogenic activity to 10-30 mcg ethinyl estradiol, for 4-15 days beginning on the day immediately following the last day of administration of the first phase combination, wherein the same dosage of the progestin and estrogen combination is administered in each of the 4-15 days, a third phase combination of a progestin at a daily dosage selected from the group consisting of 40-500 mcg trimegestone, 250 mcg-4 mg dienogest, and 250 mcg-4 mg drospirenone, and an estrogen at a daily dosage equivalent in estrogenic activity to 10-30 mcg ethinyl estradiol, for 4-15 days beginning on the day immediately following the last day of administration of the second phase combination, wherein the same dosage of the progestin and estrogen combination is administered in each of the 4-15 days, and an estrogen phase estrogen at a daily dosage equivalent in estrogenic activity to 5-30 mcg ethinyl estradiol, for 3-5 days beginning on the day immediately following the last day of administration of the third phase combination, wherein the same dosage of the estrogen is administered in each of the 3-5 days, provided that the daily dosage of the combination administered in the first phase is not the same as the daily dosage of the combination administered in the second phase and that the daily dosage of the combination administered in the second phase is not the same as the daily dosage of the combination administered in the third phase and wherein the regimen is modified to include a 25-hydroxy Vitamin D compound.

This invention further provides a method of contraception which comprises administering to a female of child bearing age a first phase of a combination of a progestin at a daily dosage equivalent in progestational activity to 40-125 mcg levonorgestrel and an estrogen at a daily dosage equivalent in estrogenic activity to 10-20 mcg ethinyl estradiol for 3-8 days beginning on day 1 of the menstrual cycle. The same daily dosage of the progestin and estrogen is administered for each of the 3-8 days. A second phase of a combination of a progestin at a daily dosage equivalent in progestational activity to 40-125 mcg levonorgestrel and an estrogen at a daily dosage equivalent in estrogenic activity to 10-20 mcg ethinyl estradiol is administered for 4-15 days beginning on the day immediately following the last day of administration of the first phase. The same daily dosage of the progestin and estrogen is administered for each of the 4-15 days. A third phase of a combination of a progestin at a daily dosage equivalent in progestational activity to 40-125 mcg levonorgestrel and an estrogen at a daily dosage equivalent in estrogenic activity to 10-20 mcg ethinyl estradiol is administered for 4-15 days beginning on the day immediately following the last day of administration of the second phase. The same daily dosage of the progestin and estrogen is administered for each of the 4-15 days. The total administration for all three phases is 23-25 days.

The invention further includes a method of contraception which comprises administering for 21 successive days to a female of childbearing age a combination of an estrogen and a progestin in a low but contraceptively effective daily dosage corresponding in estrogenic activity to 0.15-0.05 mg of 17α-ethinylestradiol and in progestogenic activity to 0.065-0.75 mg of norethindrone for 5-8 days; for the next 7-11 days an estrogen daily dosage equal to 0.02-0.05 mg of 17α-ethinylestradiol and in progestogenic activity to 0.250-1.0 mg of norethindrone; and for the next 3-7 days an estrogen daily dosage equal to 0.02-0.05 mg of 17α-ethinylestradiol and in progestogenic activity 0.35-2.0 mg of norethindrone; followed by 6-8 days without estrogen and progestogen administration, provided that the estrogen daily dosage can be the same for each period and wherein the regimen is modified so that one or more of the daily dosages further includes a 25-hydroxy Vitamin D compound. The regimen can also be modified such that one or more of the daily dosages includes a progestin dosage equivalent of at least 2.1 mg of norethindrone, preferably at least 2.5, more preferably at least 3.0, and even more preferably at least 4.0, and most preferably at least 5.0.

This invention further contemplates a method of contraception comprising the steps of sequentially-administering to a female of child bearing age: (1) for about 4 to about 7 days, a composition I containing about 0.5-1.5 mg norethindrone acetate and about 10-50 mcg ethinyl estradiol, (2) for about 5 to about 8 days, a composition II containing about 0.5-1.5 mg norethindrone acetate and about 10-50 mcg ethinyl estradiol, and (3) for about 7 to about 12 days, a composition III containing 0.5-1.5 mg norethindrone acetate and about 10-50 mcg ethinyl estradiol, wherein the amount of ethinyl estradiol is increased stepwise by the amount of at least 5 mcg in each step and wherein the regimen is modified to include a 25-hydroxy Vitamin D compound. The regimen can be modified such that one or more of the daily dosages includes at least 1.7 mg of norethindrone acetate, alternatively at least 2.0, and alternatively at least 2.5, and alternatively at least 3.0, or alternatively at least 4.0 or more.

This invention further includes contraceptive regimens which consist of the administration of a combination of a progestin (50-75 μg gestodene, 75-125 μg levonorgestrel, 60-150 μg desogestrel, 60-150 μg 3-ketodesogestrel, 100-300 μg drospirenone, 100-200 μg cyproterone acetate, 200-300 μg norgestimate, or 350-750 μg norethisterone) and an estrogen (15-25 μg EE dosage equivalent) for 23-24 days per cycle and wherein the regimen is modified to include a 25-hydroxy Vitamin D compound. The regimen can be modified such that one or more of the daily dosages includes at least 250 μg gestodene, at least 350 μlevonorgestrel, at least 400 μg desogestrel, at least 400 μg 3-ketodesogestrel, at least 750 μg drospirenone, at least 600 μg cyproterone acetate, at least 800 μg norgestimate, or at least 2.25 mg norethisterone.

This invention further contemplates triphasic progestin/estrogen combinations in which the amount of the estrogenic component is increased stepwise over the three phases. Contraceptive steroid combinations are taken for 4-7 days during the first phase (5 days being preferred); for 5-8 days during the second phase (7 days preferred); and for 7-12 days during the third phase (9 days being preferred). Following the administration of 21-days of the contraceptive steroid combination, placebo is taken for 7 days. For all three phases, 0.5-1.5 mg of norethindrone acetate is used in the progestin, with 1 mg being preferred. 10-30 μg EE is used in the first phase, 20-40 μg in the second, and 30-50 μg in the third phase and wherein the regimen is modified so that one or more of the daily dosages further includes a 25-hydroxy Vitamin D compound. The regimen can be modified such that one or more of the daily dosages includes at least 1.8 mg of norethindrone, preferably at least 2.5, more preferably 3.0, and even more preferably 4.0, and most preferably 5.0.

This invention also contemplates triphasic progestin/estrogen combination regimens in which contraceptive hormones are administered for 21 days. Contraceptive steroid combinations are taken for 5-8 days during the first phase (7 days being preferred); for 7-11 days during the second phase (7 days preferred); and for 3-7 days during the third phase (7 days being preferred). In all three phases, an estrogen at a daily dosage equivalent to 20-50 μg EE is administered in combination with a progestin having a daily dosage equivalent to 65-750 μg norethindrone in the first phase, 0.25-1.0 mg norethindrone in the second phase, and 0.35-2.0 mg norethindrone in the third phase, and wherein the regimen is modified to include a 25-hydroxy Vitamin D compound. The regimen can be modified such that one or more of the daily dosages includes at least 2.1 mg of norethindrone, preferably at least 2.5, more preferably 3.0, and even more preferably 4.0, and most preferably 5.0.

This invention also contemplates triphasic 21-day progestin/estrogen combination regimens in which a combination of 40-70 μg gestodene and an estrogen at a daily dosage equivalent of 20-35 μg EE is administered for 4-6 days in the first phase; 50-100 μg gestodene and an estrogen at a daily dosage equivalent of 30-50 μg EE is administered for 4-6 days in the second phase; and 80-120 μg gestodene and an estrogen at a daily dosage equivalent of 20-50 μg EE is administered for 9-11 days in the third phase, and placebo is administered for 7 days following the 21-day contraceptive steroid regimen; and wherein the regimen is modified to include. The regimen can be modified such that one or more of the daily dosages includes at least 200 mcg of gestodene, preferably at least 300, more preferably 600, and even more preferably 1000, and most preferably 1500.

Exemplary regimens according to this aspect of the present invention include HRT regimens with doses of progestin product less than a daily dose equivalent to 2.5 mg of medroxyprogesterone acetate daily, or less than 0.5 mg daily of a norethindrone equivalent dose. Another exemplary regimen includes a dose of progestin product greater than a daily dose equivalent to 10 mg of medroxyprogesterone acetate daily for 10-16 days every month. Exemplary regimens according to this aspect of the invention include administering progestin product at a daily dose equal to or greater than 2.5 mg daily, equal to or higher than 5 mg daily, or equal to or higher than 10 mg daily of a medroxyprogesterone equivalent dose, with a 25-hydroxy Vitamin D compound. Alternatively, a regimen useful according to the invention is that by which is administered a cumulative monthly dosage greater than the equivalent of 30 mg, 50 mg or more preferably 100 mg of medroxyprogesterone. Thus, the invention provides progestin product dosages which are greater than those currently administered on a daily and/or monthly basis.

It is contemplated that preferred HRT regimens according to one aspect of the invention would contain the lowest possible daily doses of both estrogen and progestin, but with intervening phases containing significantly higher doses of progestin in order to maximize biologic effects the 25-hydroxy Vitamin D compound.

One alternative aspect of this invention contemplates HRT regimens comprising estrogen and progestin such as Prempro where medroxyprogesterone acetate is administered at daily doses greater than 10 mg daily for some but not necessarily all of the daily dosages which include progestin, and such daily dosages may also preferably have greater than 10 mg MPA daily, and up to 20 or 30 mg daily of more for a phase of time that could last one day, one to three days, three to five days, 5-14 days or more; and with regimen cycles lasting one month, two months, three months or more. Alternatively, a more potent progestin such as levonorgestrel is substituted for provera for one or more of the days of the regimen, at doses such as at least 0.25 mg per day, more preferably at least 0.5 mg per day, most preferably at least 1 mg per day or more, for a phase of time such as that described above. The levonorgestrel is alternatively added as an additional progestin in the regimen in one or more of the daily dosages.

In some embodiments the regimen for HRT would use the lowest dosages of estrogen and progestin products possible in combination with cyclic high dosages of progestin. According to one such regimen a daily dosage of estrogen comparable to 0.325 mg to 0.625 mg conjugated estrogen, i.e. 0.010 or 0.015 mg ethinyl estradiol, plus 0.05 mg levonorgestrel is administered daily for days 1-25 followed by administration on days 26-30 of the same dosage of an estrogen product plus 0.15 or more preferably 0.25 mg or more preferably 0.5 or even more preferably 1 mg of levonorgestrel. This invention contemplates taking any of the current formulations and adding to one or more daily dosages in the regimen a 25-hydroxy Vitamin D compound. This invention further contemplates taking any one of the known HRT formulations, and changing them to contain a more potent progestin. This invention further contemplates adding higher pulses of progestin at one or more points during the one-month cycle or three-month cycle of HRT usage. This added pulse of progestin would be at amounts greater than 5 mg of medroxyprogesterone acetate or equivalents thereof per day, preferably with the pulsed amount being at least 10 mg of progestin-equivalent to medroxyprogesterone acetate or more per day, and even more preferably at dosages of 20 mg or more preferably at least 30 mg equivalent of medroxyprogesterone acetate in one or more daily dosages during the cycle. The duration of the higher pulse of progestin could be as long as one day, more preferably as long as three days, even more preferably as long as 4-10 days during a one month cycle. This invention further contemplates substituting the estrogen in the current regimens with a weaker estrogen or an estrogen having higher anti-estrogenic activity, such as tamoxifen or raloxifene.

One aspect of this invention further provides a HRT regimen which comprises a first phase comprising an estrogen at a daily dosage equivalent in estrogenic activity of 0.2-2.5 mg conjugated estrogens for 3-20 days, with estradiol, estrified estrogens, and conjugated estrogens being preferred and conjugated estrogens most preferred, and with 0.3-0.625 mg being the preferred daily dosage range and 0.3 mg most preferred, and with 10-18 days being the preferred length of the first phase and 14 days most preferred. The HRT regimen comprises a second phase comprising an estrogen at a daily dosage equivalent in estrogenic activity of 0.2-2.5 mg conjugated estrogens and a progestin at a daily dosage equivalent in progestinal activity of 2.5-10 mg medroxy-progesterone acetate for 3-20 days, with estradiol, estrified estrogens, and conjugated estrogens being preferred estrogens and conjugated estrogens most preferred, and with 0.3-0.625 mg dosage equivalent of conjugated estrogens being preferred daily estrogen dosage and 0.3 mg most preferred and with medroxy-progesterone acetate, levonorgestrel, norgestrol, and gestodene being preferred progestins, and medroxy-progesterone most preferred progestin (with 2.5 and 5 mg dosage equivalent of medroxy-progesterone acetate being most preferred), and with 10-18 days being the preferred length of the second phase and 14 days most preferred. The regimen is modified so that one or more of the daily dosages further includes a 25-hydroxy Vitamin D compound. The regimen can also be modified such that one or more of the daily dosages includes a progestin dosage equivalent of at least 0.3 mg of levonorgestrel, alternatively at least 0.5, alternatively at least 1.0, and alternatively up to 1.5 mg.

For any of the OCP regimens of this invention, the daily dosage of estrogen component, when administered, can preferably be less than 50 mcg EE dosage equivalent, and more preferably not to exceed 35 mcg, and even more preferably not to exceed 20 mcg, and most preferably not to exceed 15 mcg. For any of the OCP regimens of this invention, the daily dosage of estrogen component, when administered, can preferably be 15, 20, 25, 30, or 35 mcg EE dosage equivalent, and preferred ranges include 15-35, 15-30, 15-20, 15-20, 20-25, and 20-30 mcg EE dosage equivalent. Ethinyl estradiol (EE) is the preferred estrogen for the OC products of this invention. Other preferred estrogens include conjugated estrogens, and 17-Beta estradiol. A weaker estrogen or an estrogen having antiestrogenic activity (such as SERMs) can be used or added as a second estrogen to any of the regimens of this paragraph.

It is known that estrogen products having different affinities and activities with different estrogen receptors (ERα and Erβ) and different subspecies of those receptors can be selected to provide the desired estrogenic effects. However, the anti-estrogen products can be preferred. Thus, the preferred estrogens include selective estrogen receptor modulators (“SERM”). For example, those compounds include Clomiphene, Tamoxifen (4 OH tamoxifen), Nafoxidene, Droloxifene, Toremifene, Idoxifene, and Raloxifene. This invention contemplates using any of the above estrogens in HRT formulations of the invention, for example, as complete or partial substitutes for the estrogens in the HRT formulations identified in this application.

It is also contemplated to use dietary flavanoids in any of the embodiments having estrogens. Dietary flavanoids include phytoestrogens which are compounds found in plants that exhibit estrogenic effects on the body. There are three primary classes of phytoestrogens—isoflavones, lignans, and coumestans. Similar to estrogen, these compounds affect the central nervous system, induce estrus, and stimulate female genital tract growth. More broadly, these compounds also include chemicals that have estrogen suggestive effects including induction of specific estrogen-responsive gene products, stimulation of estrogen receptor (ER) positive breast cancer cell growth, and binding to ER's. Phytoestrogens are structurally similar to natural and synthetic estrogens and antiestrogens with diphenolic structures.

Well over 300 different types of plants have been identified as possessing sufficient estrogenic activity to induce estrus in animals. Soybeans and soy containing foods are by far the most significant dietary source of phytoestrogens, while clover, chickpeas and various other legumes, bluegrass, alfalfa, split peas, kala chana seeds, pinto bean seeds, oilseeds Such as flaxseed, dried seaweeds, and toothed medic also contain appreciable amounts. Isoflavones and coumestans are the most prevalent phytoestrogen compounds found in these and most other plants.

Isoflavones, lignans, and coumestans all include many different chemical compounds. For example, soybeans contain three main isoflavones that are each found in four chemical forms. The unconjugated forms, or aglycones, are daidzein, genistein, and glycitein. Each of these isoflavones is also found as a glucoside (daidzin, genistin, and glycitin), acetylglucoside and malonylglucoside. Processing of soy products is known to cause significant changes in the quantity and type (form) of isoflavones found in these foods. When soy flour is minimally processed it primarily contains the 6″-O-malonyladaidzin and 6″-O-malonylgenistin isomers. Further processing, such as heat-treating, will transform the malonyl isoflavones to their acetyl forms. These soy isoflavones have about one third as potent an agonist effect on the β ER as estradiol but are only 0.001 as potent as estradiol when it comes to affecting the α ER. If essence, the soy isoflavones are basically a type of selective ER modulator. Burke et al, Soybean Isoflavones as an Alternative to Traditional Hormone Replacement Therapy: Are We There Yet?, J. Nutr., 130: 664S-665S, 2000.

Although lignans have not been shown to induce estrus, they do produce other estrogen-like actions. Lignans found in humans come from the bacterial conversion of plant lignans in the gastrointestinal (GI) tract. The plant lignans, secoisolariciresinol and matairesinol, are the dietary precursors of enterodiol and enterolactone.

Isoflavones are similarly metabolized by bacteria in the GI tract. The isoflavone daidzein is metabolized to dihydrodaidzein, which is further metabolized to both equol and O-desmethylangolensin (O-DMA). Genistein is similarly metabolized to dihydrogenistein and then to 6′hydroxy-O-DMA.

Both isoflavones and lignans are absorbed and utilized through a series of conjugation/deconjugation steps with considerable variation in the actual percent metabolized depending on the individual and the type of processing that the food products have undergone. Isoflavones have been found in varying concentrations in urine, plasma, liver, lunge, kidney, brain, testis, spleen, skeletal muscle, and heart. Phytoestrogens have been shown to influence sexual differentiation, bind to the ER, affect the growth of estrogen dependent cells, affect the menstrual cycle and concentrations of reproductive formones in premenopausal women, increase vaginal cell maturation in postmenopausal women, improve cardiovascular risk factors, reduce LDL cholesterol and triglycerides, increase bone density, and potentially reduce osteoporosis associated with menopause. Kurzer, M. and Xiz Xu, Dietary Phytoestrogens, Annu. Rev. Nutr., 17:353-81, 1997. Studies have shown that countries consuming large amounts of isoflavones through soy and soy products have a markedly lower chronic disease burden than countries where relatively little soy is consumed. For example, cardiovascular disease and breast cancer mortality rates are four times lower for Japanese women than U.S. women. Endometrial cancer rates are also lower. Burke et al.

Phytoestrogens have also shown other chemopreventive activity with effects seen on leukemia and melanoma; human prostate, stomach, colon, and esophageal cancer; and rat mammary epithelial cells. This chemopreventive activity of phytoestrogens is generally believed to occur through the promotion of terminal differentiation of human tumor cells, which in turn inhibits cancer cell proliferation; inhibition of the cellular proliferation via effects on tyrosine kinases; inhibition of DNA topoisomerases' DNA replication promoting activity; enhancement of angiogenesis; antioxidant effects; and programmed cell death via apoptosis. The inhibition of cell proliferation by phytoestrogens may also involve transforming growth factor β1 signaling which includes cell specific activity and the attenuation of passage through cell cycle checkpoints via transcriptional regulation of selected proteins. Kim et al, Mechanisms of Action of the Soy Isoflavone Genistein: Emerging Role for its Effects via Transforming Growth Factor B Signaling Pathways, Am. J. Clin Nutr., 68(suppl):1418S-25S, 1998.

Although there are a large number of beneficial effects, there may also be some detrimental side effects from the consumption of large amounts of dietary phytoestrogens; for example, increasing breast cancer risk. It is still not entirely known what role these compounds play in the steroid hormone balance or whether they may compete with normal steroids and drugs. Most evidence though suggests that phytoestrogens are well tolerated. For example, there is no evidence of bleeding, breast tenderness or gastrointestinal symptoms in postmenopausal women, which when looked at in connection with the estrogen like effects of phytoestrogens has led to the suggested use of phytoestrogens in hormone replacement therapy. Phytoestrogens further include, triein, formonoetin, coumestrol, and biochanin_{A}.

This invention contemplates using any of the dietary flavanoids in any of the regimens of this invention mentioning the use of hormones, including regimens with progestins, including in single unit dosages. The invention further includes using dietary flavanoids in HRT and/or oral contraceptive formulations and regimens. The preferred daily dosage of dietary flavanoids, especially isoflavones, is at least 10 mg, with at least 20 mg being more preferred, at least 50 mg even more preferred and 80 mg even more preferred. Preferred ranges include 20-50, 20-80, and 50-80 mg of dietary flavanoids each of the specified dietary flavanoids can be include at those dosages, or mixtures thereof at those dosages. The preferred daily dosage of dietary flavanoids, especially isoflavones, is one that achieves a peak plasma level at least in the nanomolar range, or more preferably at least 1.0×10⁻⁸ molar, even more preferably at least 1.0×10⁻⁷ molar, even more preferably at least 1.0×10⁻⁶ molar, and even more preferably at least 1.0×10⁻⁵ molar.

In another alternative of the invention, a product is adapted to provide at least two different Vitamin D compounds. Preferably the product contains a 25-hydroxy Vitamin D compound with one or more other Vitamin D compounds. For example, one could provide 25-hydroxy Vitamin D3 in accordance the schedules above, but also provide Vitamin D3 or calcitriol. In one embodiment, the product is adapted to provide 25-hydroxy Vitamin D3 and calcitriol in the same composition for administration on the same day. In another embodiment, the product is adapted for the two Vitamin D compounds to be administered on different days of a regimen. In accordance with this aspect to the invention, the composition and/or regimen having at least two different Vitamin D compounds may or may not have estrogens present. Any of the formulations and/or regimens described herein containing progestins and/or estrogens may be modified to include at least two different Vitamin D compounds as described herein.

In the embodiments of the invention containing a 25-hydroxy Vitamin D compound with one or more other Vitamin D compounds, the dosage of 25-hydroxy Vitamin D could be administered at any of the dosages and scheduled described herein. In the alternative, the dosages of 25-hydroxy Vitamin D compound could be lower (e.g., one-half of the above dosages) and/or provided on a less frequent basis (e.g., one-half the number of dosages) than the dosages and schedules specified herein. For embodiments containing calcitriol, the calcitriol could be dosed at 0.25-1.0 mcg per day, with 0.5-1.0, 0.25-0.5, 0.5-1.0 being preferred and 0.5-0.75. On a weekly basis, calcitriol dosages of 2.0-10 mcg are preferred, with 2.0-6 mcg being more preferred. In order to lower the risk of hypercalcemia, the calcitriol (1,25 (OH)₂ D3) would be dosed at low levels such as 2-2.5 mcg once every week for three weeks and have the fourth week off. The fourth week could include 25 (OH) Vitamin D3. One embodiment would include 1,25 (OH)₂ D3 at therapeutic levels of 4-5 mcg once every week for three weeks and have the fourth week off from calcitriol, with 25-hydroxy Vitamin D compound at 175 mcg once a week on the same or different days as the calcitriol. Thus, one embodiment is a single unit dosage of 5 mcg calcitriol and 2.5-280 mcg 25-hydroxy Vitamin D compound, The daily dosages of 25-hydroxy Vitamin D include 2.5-40 mcg of 25-hydroxy Vitamin D, with 12.5-40 mcg, 12.5-20 mcg, 12.5-25 mcg, 25-40 mcg, 44-175 mcg, 28-140 mcg, 35-87.5 mcg, 44-87.5 mcg, 56-140 mcg, 56-140 mcg and 87.5-175 mcg of 25-hydroxy Vitamin D being preferred dosage ranges and with preferred dosages including 4 mcg, 5 mcg, 6.25 mcg, 8 mcg, 10 mcg, 12.5 mcg, 17.5 mcg, 20 mcg, 25 mcg, 28 mcg, 30 mcg, 35 mcg, 40 mcg 44 mcg, 56 mcg, 70 mcg, 88 mcg, 140 mcg, 175 mcg, and 280 mcg, with the invention including the use of dosages in ranges between such dosages.

For embodiments of the invention including at least two different Vitamin D compounds, one can administered Vitamin D without hydroxylations at the 1 or 25 positions, for example Vitamin D3 or Vitamin D2 (Non-hydroxylated Vitamin D compounds). For such Non-hydroxylated Vitamin D compounds, the preferred daily dosages range from 200-4000 IU, with ranges of 200-1000, 500-800, 1000-2000 IU preferred, and specific preferred dosages of 200, 400, 800, 1000, 1500, 2000, 3000, and 4000 IU. Alternatively, the Vitamin D compound can be administered less frequently than daily. For example, every other day, once every three days, twice a week, once a week, every two weeks and once a month. Dosages can be calculated from the daily ranges be multiplying the new frequency (e.g., double the dosages for every other day, multiply the dosages by 7 for once a week, etc.). In the alternative the dosages can remain the same even if dosed less frequently than daily. Preferred dosages include 14000 IU of Vitamin D₃ once a week or 7000 IU of Vitamin D₃ twice a week.

The terms “25-hydroxy Vitamin D” and “25(OH) Vitamin D” and “25(OH) D” includes Vitamin D compounds having a 25-hydroxylation, but without 1-alpha hydroxylation. These compounds include but are not limited to 25-hydroxyvitamin D3 and 25-hydroxyvitamin D2, and also include suitable Vitamin D analogues and derivatives with 25-hydroxylation, including fluorinated compounds. For any of the compositions and/or regimens described herein, 25-hydroxyvitamin D3 is a preferred 25-hydroxyvitamin D compound. Compounds that have 24 hydroxylation are not included in the terms “25-hydroxy Vitamin D” and “25(OH) Vitamin D” and “25(OH) D.”

The terms “progestin” and “progestin product” as used herein in the descriptions of the various embodiments of the invention includes any drug which binds to the progestin receptor and induces a progestational effect. This definition thus includes all of the known progestins, progesterones, derivatives of progesterone or testosterone that have progestin activity, progestin agonists, and progestin antagonists having a progestational effect. It is contemplated that not only presently available progestins but also progestins introduced in the future will be useful according to the present invention. The progestins include but are not limited to (a) the naturally occurring 21-carbon steroid, specifically progesterone itself and 17-hydroxyprogesterone and their derivatives; (b) the 21-carbon progesterone derivatives, specifically medroxyprogesterone, and megestrol; and (c) the 19-nortestosterone and its derivatives such as norethindrone and norgestrel. The known synthetic progestins are mainly derivatives of 17-alpha-hydroxy-progesterone or 19-nortestosterone. These progestins can be classified into three groups: the pregnane, estrane, and gonane derivatives. The pregnane progestins, derived from 17-alpha-hydroxy-progesterone, include, for example, medroxyprogesterone acetate, chlormadinone acetate, megestrol acetate, and cyproterone acetate. These are generally 20% to 50% of the potency of norethilndronie. The estranes, derived from 19-nortestosterone include norethilndrone, norethyiiodrel, norethinodryl, lynestrenol, norethindrone acetate, ethynodiol diacetate, and norethindrone enanthate. All of these are metabolized to norethindrone and are roughly equivalent to the same dosage of norethindrone. The gonanes are derived from the basic estrane structure, with the addition of an ethyl group of position 13 of the molecule. This additional ethyl group confers augmented progestogenic activity, and also significant androgenic effects. Drugs in this group include, for example, norgestrel (-d and -l), norgestimate, desogestrel, and gestodene. All of these are roughly equivalent to four times the dose of norethindrone. The progestins further include dehydrogestrone; desogestrel; 3-ketodesogestrel; dienogest; norethisterone; norethisterone acetate; progesterone; trimegestone; 19-nor-17-hydroxy progesterone ester; D-17β-acetoxy-13β-ethyl-17α-ethinyl-gon-4-en-3-one oxime; 17-hydroxyprogesterone esters and 19-nor-17-hydroxyprogesterone esters, 17α-ethinyltestosterone, 17α-ethinyl-19-nortestosterone and derivatives thereof; 17-hyroxyprogesterone, 17-hydroxyprogesterone esters, 19-nor-17-hydroxyprogesterone, 19-nor-17-hydroxyprogesterole esters, 17α-ethinyltestosterone, 17α-ethinyl-19-nortestosterone, d-17β-acetoxy-13β-ethyl-17α-ethinyl-17β-hydroxygon-4-en-3′-one, 13β-ethyl-17β-hydroxygon-4-en-3-one, 13β,17α-diethyl-17β-hydroxygon-4-en-3-one, chlormadione acetate, dimethistrone, 17α-ethinyl-β-acetoxy-19-norandrost-4-en-3 one oxime, 3-ketodesogestrel, desogestrel, gestodene, and gestodene acetate. The definition of progestin also includes newer synthetic progestins such as drospirenone, which differs from other synthetic progestins in that its pharmacological profile in preclinical studies shows it to be closer to the natural progesterone. Other new synthetic progestins are also contemplated in the use of this invention.

The term “estrogen” and “estrogen product” as used herein includes natural estrogens such as estrone, estrone sulfate, estrone sulfate piperazine salt, estradiol and estriol, and their esters, as well as ethinyl estradiol, mestranol (a 50 mg dosage of which is equivalent to 35 mg of ethinyl estradiol), conjugated equine estrogen, esterified estrogens, estropipate, 17α-ethilnylestradiol, esters and ethers of 17α-ethinylestradiol Such as, for example, 17α-ethinylestradiol 3-dimethylamino propionate, 17α-ethilnylestradiol 3-cyclopentyl ether (quinestrol) and 17α-ethinylestradiol 3-methyl ether (mestranol), estradiol-17beta, estradiol valerate, piperazine estrone sulphate, estriol succinate, and polyestrol phosphate and other estrogen equivalents and estrogen agonists and antagonists (but, as is commonly understood in the art, does not include progestins (even progestins having estrogenic activity). It is known that estrogen products having different affinities and activities with different estrogen receptors (ERα and Erβ) and different subspecies of those receptors can be selected to provide the desired estrogenic effects. However, the estrogens with anti-estrogenic activity can be preferred. Thus, the preferred estrogens include selective estrogen receptor modulators (“SERM”). For example, those compounds include Clomiphene, Tamoxifen (4 OH tamoxifen), Nafoxidene, Droloxifene, Toremifene, Idoxifene, and Raloxifene. The estrogens also include phytoestrogens, including Isoflavones. This invention contemplates using any of the above estrogens in HRT formulations of the invention, for example, as complete or partial substitutes for the estrogens in the HRT formulations identified in this application.

According to a preferred aspect of the invention, the 25-hydroxy Vitamin D compound and a progestin may be coadministered as a pharmaceutical composition preferably in a single unit dosage, such as a tablet, for inhibiting the conversion of non-neoplastic ovarian epithelial cells to neoplastic cells. “Concurrent administration” or “co-administration” as used herein includes administration of the agents together, or before or after each other. The agents may be administered by different routes. For example, one agent may be administered intravenously while the second agent is administered intramuscularly, intravenously or orally, or via a patch, gel or implant that can be placed on or in the skin or in the vagina or uterus. They may be administered simultaneously or sequentially, as long as they are given in a manner sufficient to allow both agents to achieve effective concentrations in the body. The preferred manner of co-administration for all the combinations described above is a single unit dosage, such as a single tablet.

The invention includes the use of pill packs to enhance compliance of OCs and HRTs with Vitamin D. The pill pack could be for 28 days, or a shorter length of time, such as 21 days, or longer number of days, such as 91 days. In one embodiment, the pill pack has 28 pills with 1-28 of the pills having 25-hyroxy Vitamin D3 in the dosages described herein. The estrogen and progestin levels in the 28 pills would be inaccordance with the regimens described herein. Thus, in one mono-phasic OC, the pill pack would contain 21 pills with estrogen, progestin and Vitamin D in each pill and 7 pills with Vitamin D only (the placebo week). In one tri-phasic regimen, the pill pack would include 21 or 28 pills like a normal pill pack, but with the 7 pills with the highest progestin level also including 25-hyroxy Vitamin D3. These are just exemplary. HRT regimens would include, for example, pill packs with Vitamin D added to all the pills. Alternatively, the pill pack could include 25-hyroxy Vitamin D3 every 7th pill, or just on days when progestin is administered, for example for 14 consecutive days in one HRT regimen and for 3 consecutive days in another HRT regimen.

Alternatively, the product formulations could have the Vitamin D in a separate pill from the pill containing progestin and/or estrogen. The pill pack could have configurations as known in art, for example rows of pills, The rows could include a modification of an additional row for a second pill of Vitamin D for each day. Alternatively, where the Vitamin D is not administered each day, the the rows would include a second pill outside the hormone row of pills adjacent to one or more of the of the hormone pills. In the alternative, one could use a circular arrangement, with for example 28 pills. The circular pack for an OC could include 21 pills with progestin and/or estrogen, at the levels for the OC formulations discussed herein, and 7 placebo pills. The circular pack could include a second ring of pills adjacent (outer or inner) the hormone ring of pills. The second ring would include the Vitamin D compound. The ring pack could include 28 pills with 25-hyroxy Vitamin D3 every day, or less frequently, such as twice a week, once every week (4 pills), once every two weeks (2 pills), or once a cycle (1 pill or more than one pill lined up for that day). For the arrangement where there are less Vitamin D pills, that ring should be inside the ring of hormone pills. If two rings of 28 pills are desired, the Vitamin D ring could include placebo pills for regimens where Vitamin D is administered less frequently than daily.

Alternatively, for an HRT, the pack could include 28 pills with estrogen, and some of those pills with progestin, at the levels for the HRT formulations discussed herein. In one embodiment, the pack also includes 14 tablets of 25-hyroxy Vitamin D3 for administration every other day. The pack could also include 25-hyroxy Vitamin D3 only when progestin is administered for an HRT product, or when the progestin is the highest in a phasic OC. Although this invention contemplates 25-hyroxy Vitamin D as the preferred Vitamin D compound, if one were to use regular Vitamin D or calcitriol, or other Vitamin D compounds, one could use the inventive aspects of using the pill pack arrangements described herein (in any configuration) to improve compliance. In HRTs, one could make the pill packs used commonly with OCs to have increased compliance of the HRT with the Vitamin D compound.

All doses given herein are based generally for a female subject of about 60 kg weight; the dosages naturally can vary more or less depending on the weight of the subject, although the dosage can be the same for women in general as it is in many HRT and OC products. The doses may be increased or decreased, and the duration of treatment may be shortened or lengthened as determined by the treating physician. The frequency of dosing will depend on the pharmacokinetics parameters of the agents and the route of administration. The optimal pharmaceutical formulation will be determined by one skilled in the art depending upon the route of administration and desired dosage. See for example, Remington's Pharmaceutical Sciences, 18th Ed. (1990, Mack Publishing Co., Easton, Pa. 18042) pages 1435-1712, the disclosure of which is hereby incorporated by reference. Such formulations may influence the physical state, stability, rate of in vivo release, and rate of in vivo clearance of the administered agents.

It is contemplated that the routes of delivery of Vitamin D compounds including Vitamin D and biologically active analogues and derivatives thereof (either alone or in combination with other pharmaceuticals) could include oral, sublingual, injectable (including short-acting, depot, implant and pellet forms injected subcutaneously or intramuscularly), vaginal creams, suppositories, pessaries, rings, rectal suppositories, intrauterine devices, and transdermal forms such as patches and creams.

### EXAMPLE 1

### Tests on Ovarian Epithelial Cell Viability

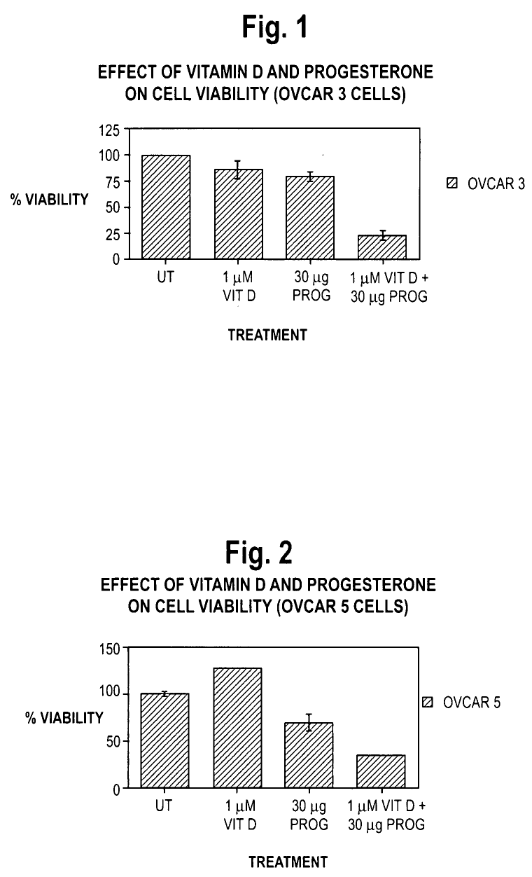

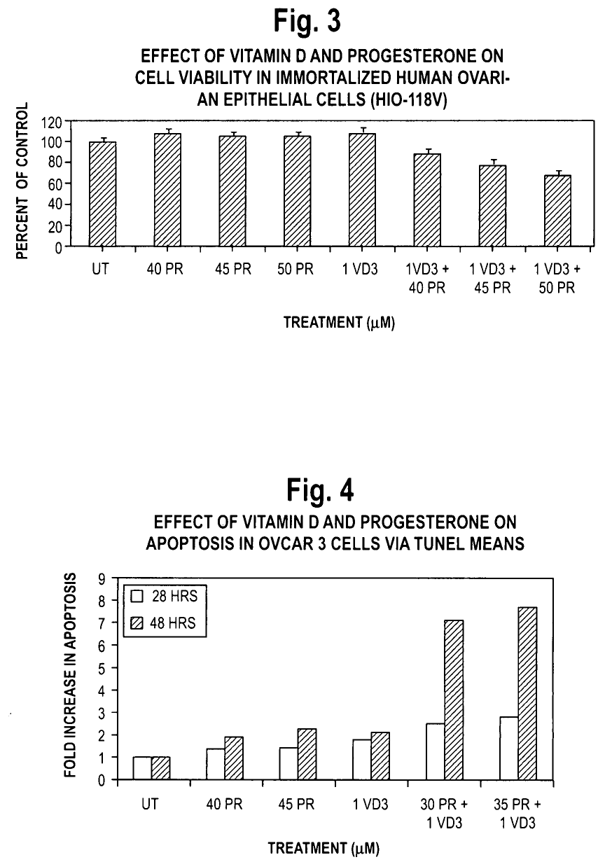

Progesterone and Vitamin D (1,25 (OH)2 D3) were tested to determine their effect on cell lines derived from the human ovarian surface epithelium. FIGS. 1-3 show the effect on programmed cell death in cell lines derived from the human ovarian surface epithelium for Untreated (UT), Progesterone, Vitamin D (1,25 (OH)2 D3), and the combination of progestin and vitamin D. Both progestin and vitamin D inhibit cell viability in a dose response fashion. MTS assays evaluating the combination of progestin and vitamin D demonstrate that combining the two agents confers a dramatically more potent inhibitory effect on cell viability than either agent alone. This is shown in FIGS. 1-3 in both ovarian cancer cell lines (OVCAR 5 and OVCAR 3) as well as an immortalized cell cultures derived from the normal human ovarian epithelium (HIO-118V). There is a marked impact on cell viability when the two agents are combined and administered at a dosage that has a marginal impact for each agent given alone.

The data have been analyzed isobolographically to determine if the drug combinations are acting additively or synergistically. For these analyses we have used the CalcuSyn software (Biosoft). Raw data for each drug or drug combination dose are entered singly to generate a median effect plot. From this plot, the combination index (CI) is generated to determine whether the drug effects were additive, synergistic or antagonistic. CI values of <1, =1 or >1 indicate synergy, additivity or antagonism, respectively. The data demonstrate Cl values significantly less than one, indicating that that the combination of a progestin and Vitamin D act synergistically to inhibit ovarian epithelial cell viability.

### EXAMPLE 2

### Apoptotic Effect of Progestin and Vitamin D

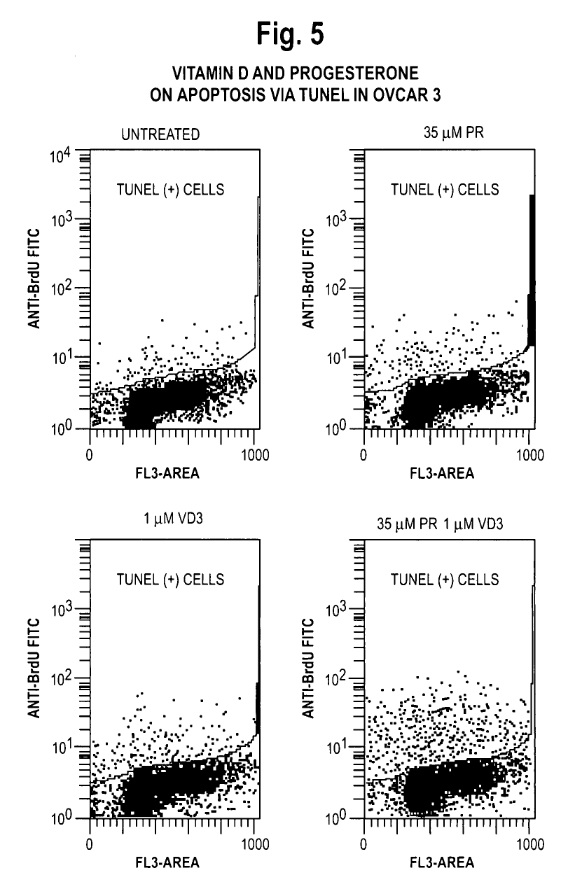

In experiments to further understand the effect of progestin and vitamin D on cell viability, experiments were conducted to determine how progestin, vitamin D, and the combination act to induce apoptosis. In the experiment cells were incubated for 28 and 48 hours in the hormonal treatments as indicated and assessed for TUNEL reactivity. In these experiments, Apoptosis (TUNEL) data shown in conditions in which inhibitory effects via MTS were shown. In the example shown in FIG. 4, OVCAR 3 cells undergo a 7-fold increase in apoptosis at 48 hrs when treated with a combination of progesterone and vitamin D. HIO-118V cells show a 1.7-fold increase in apoptosis with 45 uM progesterone alone and a 1.9-fold increase with the combination of 1 uM Vitamin D and 45 uM progesterone. FIG. 5 shows the (TUNEL) data for the OVCAR 3 cells, with the combination of progesterone and vitamin D having the most significant amount of apoptosis.

### EXAMPLE 3

### Effects of Progestin on Vitamin D 24 Hydroxylase

Tests were later conducted in an effort to search for molecular mechanisms underlying the synergistic effect of Progestins and Vitamin D on the ovarian epithelium. The Vitamin D metabolizing enzyme 24 hydroxylase (24-OH) converts the active form of Vitamin D (1,25 (OH)2 D) to an inactive form via 24 hydroxylation. Of note, many cancer cells over-express 24-OH, rendering them resistant to the effects of Vitamin D. Moreover, 24-OH is normally induced in cells in response to Vitamin D. This serves to inhibit unbridled Vitamin D effects and to turn off Vitamin D once it has achieved its biologic effect. Agents which inhibit 24-OH have the potential to enhance the biologic effect of vitamin D by inhibiting its degradation, and thus increasing its half life. Previously, Genistein had been shown by others to inhibit 24-OH.

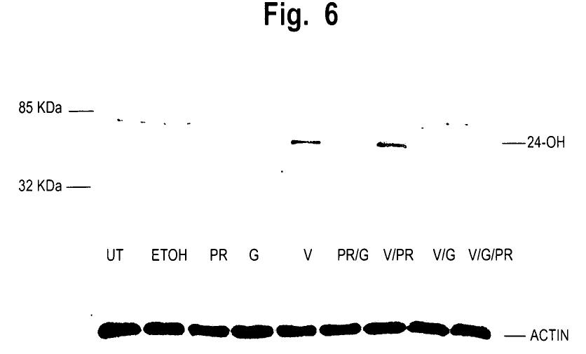

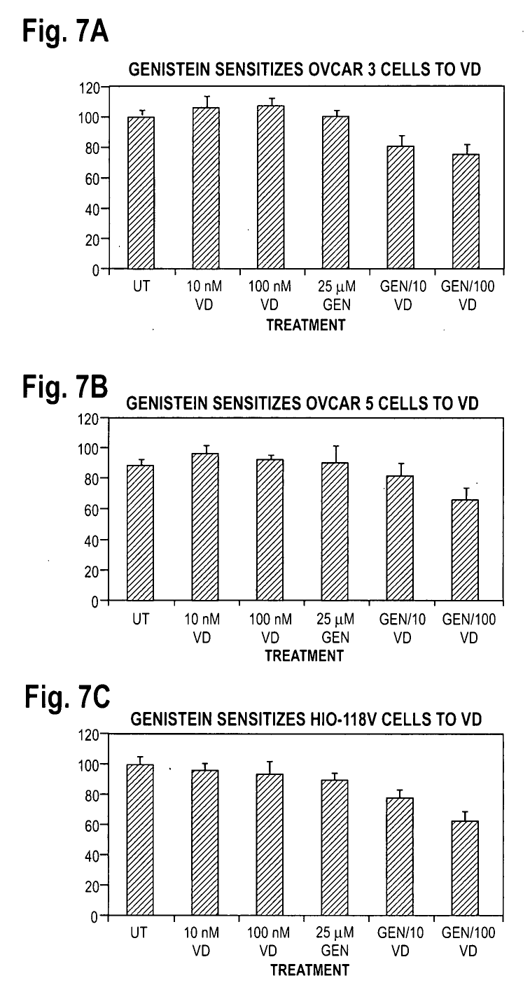

OVCAR 3 cells were incubated for 18 hours in various conditions, including untreated control and vehicle control (ut and ETOH) as well as with vitamin D (V), Genistein (G), Progesterone (PR) and combinations of Genistein, vitamin D, and progesterone. Protein was then extracted from the cells and 24-OH expression was the measured via Western Blot, and the degree of expression (band density) measured via a densitometer. Of note, 24-OH activity is markedly induced as expected by vitamin D. As shown in FIG. 6, the addition of 25 μM progesterone (PR) and/or 25 μM Genistein (G) in combination with 100 nM Vitamin D (V) significantly decreases expression of 24-OH. Of the apparent band at the level of 24-OH in the V/PR lane is actually two bands, which suggests that 24-OH may be degraded by progesterone, possibly causing an inactive splice variant of 24-OH. This effect of a progestin on 24-OH has not been previously shown, but may explain in part the synergy associated with the progestin-Vitamin D combination. Namely, by inhibiting Vitamin D's inactivation via degradation and inhibition of 24-OH, the active form of Vitamin D has a longer local biologic half life, and thus cellular effect when combined with progestin. The addition of Genistein to progestin and vitamin D inhibits 24-OH even further. These data are further supported by the experiments shown in FIGS. 7a-c (MTS assay). Pretreatment of ovarian cancer cell lines with Genistein enhances the inhibitory effect of vitamin D on cell viability.

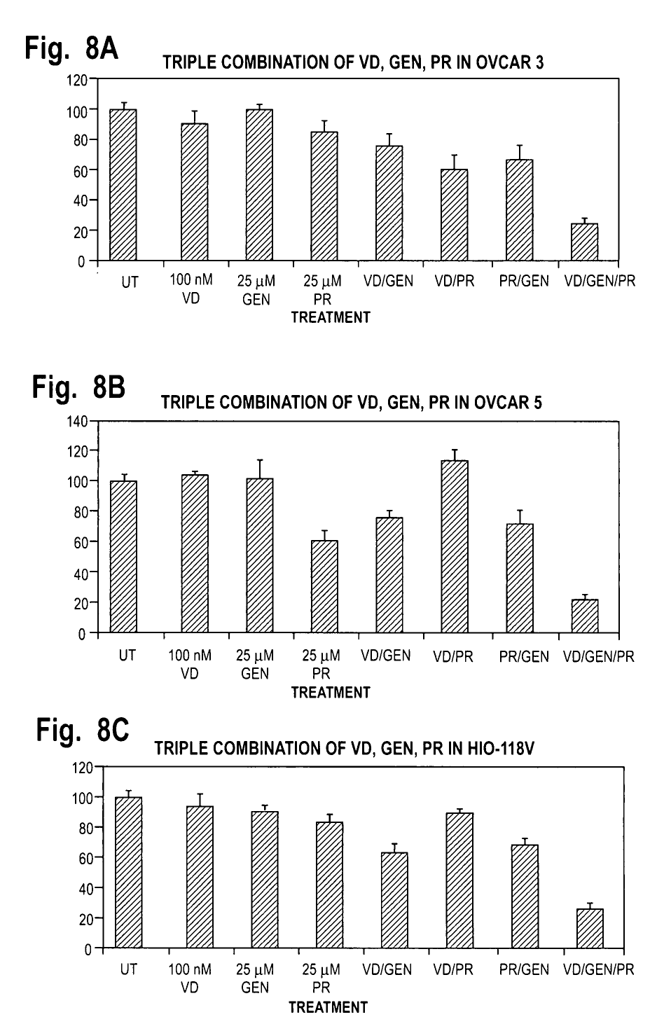

Next, as shown in FIGS. 8a-c, 24 hour pretreatment with Genistein followed by the combination treatment of Vitamin D and progesterone revealed very pronounced killing of the ovarian cell lines. Of note, this finding occurred concomitant with the western blot data demonstrating marked reduction of 24-OH with the progestin and Genistein combination. In addition, it is possible that other estrogens (both plant and non-plant-derived), in addition to the phytoestrogen genistein, could confer the same benefit.

The data shown above demonstrate synergistic activation of cell death in cells derived from the ovarian surface epithelium by the combination of progestin and Vitamin D. Progestin decreases the degradation of Vitamin D via tile decreasing expression and possible degradation of the enzyme 24-OH, which thus leads to decreased metabolic inactivation of the active form of Vitamin D (1,25 (OH)2 D), thereby increasing its potency. The inventor hypothesizes that progestins and Vitamin D target the early steps of carcinogenesis in the ovarian epithelium, by activating pathways leading to apoptosis and thereby decreasing dysplastic ovarian epithelial cells in the non malignant ovarian epithelium, resulting in effective cancer prevention. In addition, the inventor hypothesizes the addition of a progestin to 25-hydroxt vitamin D will provide a novel approach for increasing local vitamin D potency in the ovarian epithelium while not requiring increased systemic dosing of vitamin D, with the associated adverse systemic side effects of high dosages of vitamin D.

### EXAMPLE 4

### Prophetic

An OC product contain 28 daily dosages in the form of 28 pills, with (1) daily dosages 1-7 including 35 mcg Ethinyl estradiol and 0.18 mg norgestimate, (2) daily dosages 8-14 including 35 mcg Ethinyl estradiol and 0.215 mg norgestimate, and (3) daily dosages 15-21 including 35 mcg Ethinyl estradiol, 0.25 mg norgestimate, and 40 mcg 25-hydroxy Vitamin D3. Daily dosages 22-28 contain placebo.

### EXAMPLE 5

### Prophetic

An OC product contain 28 daily dosages in the form of 28 pills, with (1) daily dosages 1-7 including 35 mcg Ethinyl estradiol and 0.18 mg norgestimate, (2) daily dosages 8-14 including 35 mcg Ethinyl estradiol and 0.215 mg norgestimate, and (3) daily dosages 15-21 including 35 mcg Ethinyl estradiol and 0.25 mg norgestimate. Daily dosages 22-28 contain no progestin or estrogen. Daily dosages 7, 14, 21, and 28 further each include 70 mcg 25-hydroxy Vitamin D3.

### EXAMPLE 6

### Prophetic

An OC product contain 28 daily dosages in the form of 28 pills, with (1) daily dosages 1-7 including 25 mcg Ethinyl estradiol and 0.18 mg norgestimate, (2) daily dosages 8-14 including 25 mcg Ethinyl estradiol and 0.215 mg norgestimate, and (3) daily dosages 15-21 including 25 mcg Ethinyl estradiol and 0.25 mg norgestimate. Daily dosages 22-28 contain no progestin or estrogen. All 28 daily dosages further each include 20 mcg 25-hydroxy Vitamin D3.

### EXAMPLE 7

### Prophetic

An OC product comprises tablets each containing 90 microgram levonorgestrel, 20 microgram ethinyl estradiol, and 10 mcg 25-hydroxy Vitamin D3.

What is claimed is:

1. A composition comprising 25-hydroxy Vitamin D compound and a progestin, wherein the dosage of said Vitamin D compound is in the range of 41-560 mcg.

2. The composition of claim 1 wherein the dosage of said Vitamin D compound is at least 50 mcg.

3. The composition of claim 1 wherein the dosage of said Vitamin D compound is in the range of 41-87.5 mcg.

4. The composition of claim 1 wherein the dosage of said Vitamin D compound is in the range of 44-87.5 mcg.

5. The composition of claim 1 wherein the dosage of said Vitamin D compound is in the range of 56-140 mcg.

6. The composition of claim 1 wherein the dosage of said Vitamin D compound is in the range of 87.5-175 mcg.

7. The composition of claim 1 wherein the dosage of said Vitamin D compound is in the range of 200-400 mcg.

8. The composition of claim 1 wherein said Vitamin D compound is 25-hydroxy Vitamin D₃.

9. The composition of claim 1 wherein said Vitamin D compound is 25-hydroxy Vitamin D₂.

10. The composition of claim 9 wherein the dosage of said Vitamin D compound is in the range of 41-100 mcg.

11. The composition of claim 9 wherein the dosage of said Vitamin D compound is in the range of 44-175 mcg.

12. The composition of claim 1 further including an estrogen compound.

13. The composition of claim 12 wherein said estrogen compound is a conjugated equine estrogen.

14. The compound of claim 12 wherein said estrogen compound is estradiol.

15. The composition of claim 1 wherein said progestin is norgestimate.

16. The composition of claim 15 wherein the dosage of said progestin is 0.18-0.25 mg norgestimate.

17. The composition of claim 16 further including 15-35 mcg ethinyl estradiol.

18. The composition of claim 1 wherein said progestin is levonorgestrel and further includes ethinyl estradiol.

19. The composition of claim 18 wherein said hormone compound includes 90 mcg levonorgestrel and 20 mcg ethinyl estradiol.

20. The composition of claim 1 wherein said progestin is 3.0 mg drospirenone and further includes 20-30 mcg ethinyl estradiol.

21. A pharmaceutical combination comprising 21 daily sequential dosages, said product including 21 sequential daily hormone dosages, with at least one hormone dosage comprising a progestin product, and optionally estrogen, and wherein the remaining daily hormone dosages include estrogen, and optionally a progestin product, and with at least one of said dosages further comprising a 25-hydroxy Vitamin D compound in the range of 35-560 mcg, and at least another one of said hormone dosages has Vitamin D in the range of 0-10 mcg.

22. The combination of claim 21 wherein the dosage of said Vitamin D compound is at least 41 mcg.

23. The combination of claim 21 wherein the dosage of said Vitamin D compound is in the range of 35-87.5 mcg.

24. The combination of claim 21 wherein the dosage of said Vitamin D compound is in the range of 44-87.5 mcg.

25. The combination of claim 21 wherein the dosage of said Vitamin D compound is in the range of 56-140 mcg.

26. The combination of claim 21 wherein the dosage of said Vitamin D compound is in the range of 87.5-175 mcg.

27. The combination of claim 21 wherein the dosage of said Vitamin D compound is in the range of 200-400 mcg.

28. The combination of claim 21 wherein said Vitamin D compound is 25-hydroxy Vitamin D₃.

29. The combination of claim 21 wherein said Vitamin D compound is 25-hydroxy Vitamin D₂.

30. The combination of claim 21 wherein said at least another one of said hormone dosages has no Vitamin D.

31. The combination of claim 21 wherein said at least one of said dosages further comprising a 25-hydroxy Vitamin D compound in the range of 35-560 mcg includes no more than 14 of the 21 dosages.

32. The combination of claim 21 wherein said at least one of said dosages further comprising a 25-hydroxy Vitamin D compound in the range of 35-560 mcg includes no more than 11 of the 21 dosages.

33. The combination of claim 21 wherein said at least one of said dosages further comprising a 25-hydroxy Vitamin D compound in the range of 35-560 mcg includes no more than 6 of the 21 dosages.

34. The combination of claim 21 wherein said estrogen product is ethinyl estradiol.

35. The combination of claim 34 wherein the daily dosage of said ethinyl estradiol is less than or equal to 0.035 mg and greater than or equal to 0.015 mg.

36. The combination of claim 21 wherein said progestin product is a gonane.

37. The combination of claim 21 wherein said progestin product is a pregnane.

38. The combination of claim 21 wherein said progestin product is a estrane.

39. The combination of claim 21 wherein said progestin product is norgestimate in the range of 0.18-0.25 mcg.

40. The combination of claim 39 wherein said the estrogen product in at least one hormone dosage is ethinyl estradiol and the daily dosage of said ethinyl estradiol is less than or equal to 0.035 mg and greater than or equal to 0.020 mg.

41. The combination of claim 21 wherein at least one Vitamin D compound is in a single unit dosage with one dosage of said progestin product.

42. The combination of claim 21 wherein at least one hormone dosage includes 3.0 mg drospirenone and 20-30 mcg ethinyl estradiol.

43. A pharmaceutical combination comprising 28 daily sequential dosages, said product including 21 sequential daily hormone dosages, with at least one hormone dosage comprising a progestin product, and optionally estrogen, and wherein the remaining daily hormone dosages include estrogen, and optionally a progestin product, and with at least one of said dosages further comprising a 25-hydroxy Vitamin D compound in the range of 35-560 mcg, and at least another one of said hormone dosages has Vitamin D in the range of 0-10 mcg, and said combination further comprising 7 additional daily sequential dosages, wherein said additional dosages may include progestin and/or estrogen, or alternatively have no estrogen and no progestins, and wherein at least one of said additional dosages includes a 25-hydroxy Vitamin D compound in the range of 35-560 mcg.

44. The combination of claim 43 wherein said product is adapted for administration of said Vitamin D compound once every 7 daily sequential dosages.

45. The combination of claim 44 wherein said product is adapted for administration of said Vitamin D compound in less than 15 of said daily sequential dosages.

46. The combination of claim 44 wherein said product is adapted for administration of said Vitamin D compound in less than 9 of said daily sequential dosages.
